(19) United States
(12) Patent Application Publication
(10) Pub. No.: US 2019/0083034 A1
(43) Pub. Date: Mar. 21, 2019

(54) WATCH-TYPE TERMINAL AND METHOD FOR CONTROLLING SAME

(71) Applicant: LG ELECTRONICS INC., Seoul (KR)

(72) Inventors: Hongjo SHIM, Seoul (KR); Jisoo PARK, Seoul (KR); Youngho SOHN, Seoul (KR); Hyunok LEE, Seoul (KR); Jeonghan KIM, Seoul (KR); Mihyun PARK, Seoul (KR)

(73) Assignee: LG ELECTRONICS INC., Seoul (KR)

(21) Appl. No.: 16/095,237

(22) PCT Filed: Nov. 24, 2016
(86) PCT No.: PCT/KR2016/013655

§ 371 Date: Oct. 19, 2018

Related U.S. Application Data
(60) Provisional application No. 62/328,624, filed on Apr. 28, 2016.

(30) Foreign Application Priority Data
Jul. 27, 2016 (KR) 10-2016-0095637

(51) Int. Cl. A61B 5/00; A61B 5/1455

(52) U.S. Cl. CPC A61B 5/681; A61B 5/14552; A61B 5/4818; A61B 5/746; A61B 5/14546; A61B 5/486; A61B 5/4812; A61B 5/0004; A61B 5/7278; A61B 5/7425

(57) ABSTRACT

A watch-type terminal including a main body; a sensing unit disposed on the main body; a display unit; and a controller. Further, the sensing unit includes at least one green light-emitting element to output green light; a light-receiving sensor spaced apart from the at least one green light-emitting element to receive green light reflected from one part of the human body; a red light-emitting element spaced apart from the light-receiving sensor to output red light; and an infrared (IR) sensor spaced apart from the light-receiving sensor to output IR light. In addition the controller calculates an oxygen saturation of blood in a human body wearing the watch-type terminal based on an oxygen absorbance of hemoglobin in the human body through reflectance of the red light and the IR light.

### CROSS-REFERENCE TO RELATED APPLICATIONS

This application is the National Stage filing under 35 U.S.C. 371 of International Application No. PCT/KR2016/013655 filed on Nov. 24, 2016, which claims the benefit of earlier filing date and right of priority to Korean Application No. 10-2016-0095637 filed in the Republic of Korea on Jul. 27, 2016 and right of priority to U.S. Provisional Application No. 62/328,624 filed on Apr. 28, 2016, the contents of which are all hereby incorporated by reference herein in their entirety.

### FIELD

The present invention relates to a watch-type terminal in which a specific function is controlled by sensing a worn state of the terminal.

### BACKGROUND

Terminals may be divided into glass-type terminals and stationary terminals according to mobility. Also, the glass-type terminals may be classified into handheld types and vehicle mount types according to whether or not a user can directly carry.

As it becomes multifunctional, a terminal can be allowed to capture still images or moving images, play music or video files, play games, receive broadcast and the like, so as to be implemented as an integrated multimedia player. Efforts are ongoing to support and increase the functionality of terminals. Such efforts include software improvements, as well as changes and improvements in the structural components.

As a wearable terminal mounted on a part of a human body is developed, various functions are implemented, and a security function is also improved by activating or restricting a specific function in a manner of sensing whether a user wears the wearable terminal. As the wearable terminal mounted on the part of the human body is developed, a sensing module for recognizing a breathing state using the wearable terminal is being studied. However, there is a disadvantage in that an accurate measurement is difficult due to a small size of the wearable terminal, a lot of movements in a worn state on the human body, and a condition or feature of a worn body portion.

### SUMMARY

Accordingly, an aspect of the present invention is to provide a watch-type terminal having a sensing unit provided with a light-receiving sensor and a light-emitting element, which are spaced apart from each other to maintain a specific distance for accurate measurement of a biological signal.

To achieve this aspect and other advantages, a watch-type terminal according to one embodiment of the present invention may include a main body, a sensing unit disposed on one surface of the main body to acquire a biological signal, and a controller (or a control unit). The sensing unit may include at least one green light-emitting element disposed on one surface of the main body to output green light, a light-receiving sensor disposed to be spaced apart from the green light-emitting element to receive green light reflected from one part of a human body, a red light-emitting element disposed to be spaced apart from the light-receiving sensor to output red light, and an IR sensor disposed to be spaced apart from the IR light-receiving sensor to output IR light. The controller may calculate oxygen saturation based on an oxygen absorbance of hemoglobin through reflectance of the red light and the IR light.

In one embodiment related to the present invention, the controller may transmit sleep state information based on the oxygen saturation to a preset external device to control a function of the external device. Therefore, it is possible to control the function of a linked external device of a user, or to provide guide information to a counterpart located adjacent to the user.

In one embodiment of the present invention, guide information may be output or an execution of a specific function may be controlled based on the sleep state information, prestored information and/or sensing information sensed by the sensing unit. This may result in predicting the user's state by a sleep state and performing a function based on the predicted result.

According to the present invention, since a light-emitting element and a light-receiving sensor are disposed separately, a red light-emitting element and an IR sensor can be disposed apart from the light-receiving sensor by a specific distance or more. Therefore, oxygen saturation according to reflectance of red light and IR light can be measured.

Also, since a mobile terminal is controlled based on breathing state information based on the oxygen saturation, the user can be guided to take a proper sleep, or his/her life and the use of the terminal can be facilitated in a state of insufficient sleep.

In addition, since breathing state information can be transmitted to an external device, another linked terminal of the user can be controlled according to the user's state even during use of the another terminal. Also, since the breathing state information can be transmitted to a terminal of another user, guide information which is helpful for the other user's life or the user's health can be managed.

### BRIEF DESCRIPTION OF THE DRAWINGS

FIG. 1A is a block diagram of a watch-type terminal in accordance with one embodiment of the present invention.

FIG. 1B is a view of a watch-type terminal according to one embodiment viewed from one direction.

FIG. 1C is a conceptual view of a watch-type terminal according to one embodiment of the present invention, viewed from one direction.

FIG. 2A is a conceptual view illustrating a configuration and an arrangement structure of a sensing module.

FIG. 2B is a graph illustrating a light absorption rate of hemoglobin (Hb) and oxygen hemoglobin (HbO2) according to a wavelength of light.

FIGS. 3A to 3C are conceptual views illustrating a sensing unit for outputting red light for measuring an oxygen saturation.

FIGS. 4A to 4D are conceptual views illustrating a sensing unit that outputs red light for measuring an oxygen saturation according to another embodiment.

FIGS. 5A to 5G are conceptual views illustrating a sensing unit which includes two light-receiving sensors and is capable of measuring an oxygen saturation.

FIG. 6A is a flowchart illustrating a method of controlling a mobile terminal using an oxygen saturation detected by a sensing unit of the present invention.

FIG. 6B is a conceptual view illustrating the control method of FIG. 6A.

FIGS. 7A and 7B are conceptual views illustrating a method of controlling a watch-type terminal and/or a mobile terminal performing wireless communication with the watch-type terminal, in accordance with one embodiment of the present invention.

FIGS. 8A to 8C are conceptual views illustrating a control method for providing guide information based on stored information and sleep state information.

FIGS. 9A to 9C are conceptual views illustrating a control method for providing guide information analyzed through collected sleep state information and additional information.

FIGS. 10A and 10B are conceptual views illustrating a control method in a state where a warning mode is activated.

FIGS. 11A to 11E are conceptual views illustrating a method of controlling a watch-type terminal and an external device cooperating with the watch-type terminal, in accordance with another embodiment.

### DETAILED DESCRIPTION OF THE EMBODIMENTS

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In describing the present disclosure, if a detailed explanation for a related known function or construction is considered to unnecessarily divert the gist of the present disclosure, such explanation has been omitted but would be understood by those skilled in the art. The accompanying drawings are used to help easily understand the technical idea of the present disclosure and it should be understood that the idea of the present disclosure is not limited by the accompanying drawings. The idea of the present disclosure should be construed to extend to any alterations, equivalents and substitutes besides the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element can be connected with the other element or intervening elements may also be present. In contrast, when an element is referred to as being “directly connected with” another element, there are no intervening elements present.

A singular representation may include a plural representation unless it represents a definitely different meaning from the context. Terms such as “include” or “has” are used herein and should be understood that they are intended to indicate an existence of several components, functions or steps, disclosed in the specification, and it is also understood that greater or fewer components, functions, or steps may likewise be utilized.

Mobile terminals presented herein may be implemented using a variety of different types of terminals. Examples of such terminals include cellular phones, smart phones, user equipment, laptop computers, digital broadcast terminals, personal digital assistants (PDAs), portable multimedia players (PMPs), navigators, portable computers (PCs), slate PCs, tablet PCs, ultra books, wearable devices (for example, smart watches, smart glasses, head mounted displays (HMDs)), and the like.

By way of non-limiting example only, further description will be made with reference to particular types of mobile terminals. However, such teachings apply equally to other types of terminals, such as those types noted above. In addition, these teachings may also be applied to stationary terminals such as digital TV, desktop computers, and the like.

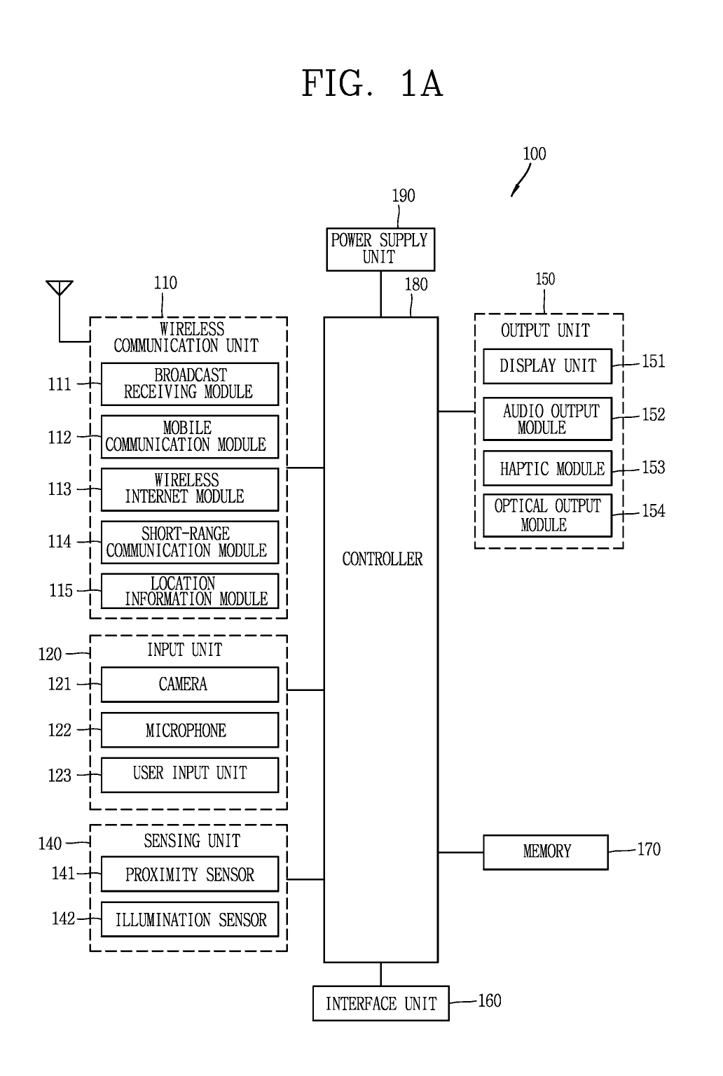

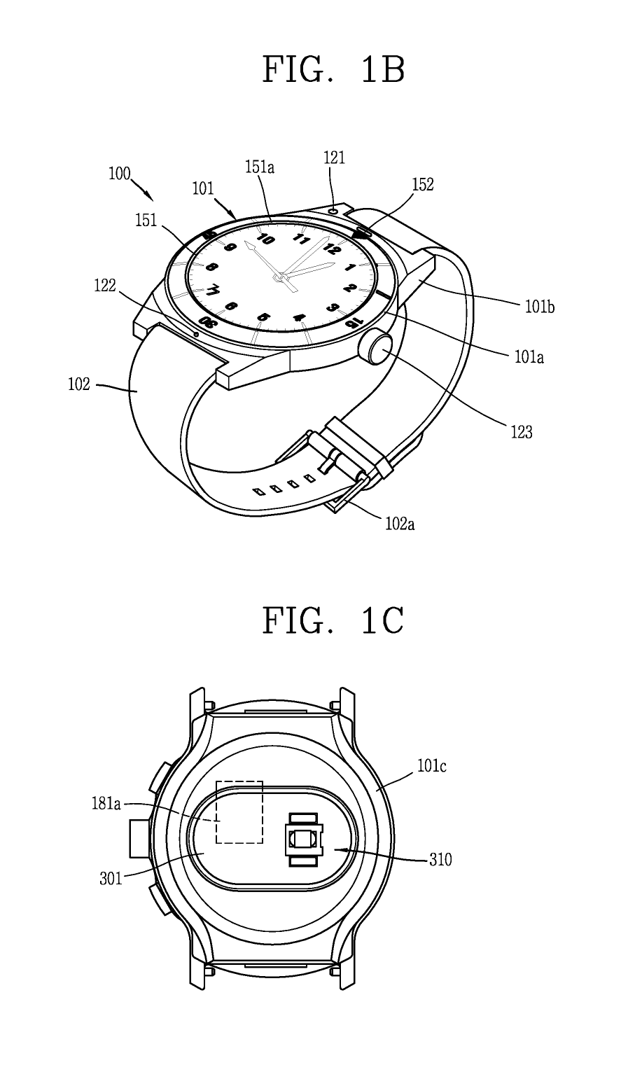

FIG. 1A is a block diagram of a mobile terminal in accordance with one exemplary embodiment of the present invention. The mobile terminal 100 may be shown having components such as a wireless communication unit 110, an input unit 120, a sensing unit 140, an output unit 150, an interface unit 160, a memory 170, a controller 180, and a power supply unit 190. It is understood that implementing all of the illustrated components is not a requirement, and that greater or fewer components may alternatively be implemented.

In more detail, the wireless communication unit 110 may typically include one or more modules which permit communications such as wireless communications between the mobile terminal 100 and a wireless communication system, communications between the mobile terminal 100 and another mobile terminal, or communications between the mobile terminal 100 and an external server. Further, the wireless communication unit 110 may typically include one or more modules which connect the mobile terminal 100 to one or more networks.

The wireless communication unit 110 may include one or more of a broadcast receiving module 111, a mobile communication module 112, a wireless Internet module 113, a short-range communication module 114, and a location information module 115.

The input unit 120 may include a camera 121 or an image input unit for obtaining images or video, a microphone 122, which is one type of audio input device for inputting an audio signal, and a user input unit 123 (for example, a touch key, a mechanical key, and the like) for allowing a user to input information. Data (for example, audio, video, image, and the like) may be obtained by the input unit 120 and may be analyzed and processed according to user commands.

The sensing unit 140 may typically be implemented using one or more sensors configured to sense internal information of the mobile terminal, the surrounding environment of the mobile terminal, user information, and the like. For example, the sensing unit 140 may include at least one of a proximity sensor 141, an illumination sensor 142, a touch sensor, an acceleration sensor, a magnetic sensor, a G-sensor, a gyroscope sensor, a motion sensor, an RGB sensor, an infrared (IR) sensor, a finger scan sensor, a ultrasonic sensor, an optical sensor (for example, camera 121), a microphone 122, a battery gauge, an environment sensor (for example, a barometer, a hygrometer, a thermometer, a radiation detection sensor, a thermal sensor, and a gas sensor, among others), and a chemical sensor (for example, an electronic nose, a health care sensor, a biometric sensor, and the like). The mobile terminal disclosed herein may be configured to utilize information obtained from one or more sensors of the sensing unit 140, and combinations thereof.

The output unit 150 may typically be configured to output various types of information, such as audio, video, tactile output, and the like. The output unit 150 may be shown having at least one of a display unit 151, an audio output module 152, a haptic module 153, and an optical output module 154. The display unit 151 may have an inter-layered structure or an integrated structure with a touch sensor in order to implement a touch screen. The touch screen may function as the user input unit 123 which provides an input interface between the mobile terminal 100 and the user and simultaneously provide an output interface between the mobile terminal 100 and a user.

The interface unit 160 serves as an interface with various types of external devices that are coupled to the mobile terminal 100. The interface unit 160, for example, may include any of wired or wireless ports, external power supply ports, wired or wireless data ports, memory card ports, ports for connecting a device having an identification module, audio input/output (I/O) ports, video I/O ports, earphone ports, and the like. In some cases, the mobile terminal 100 may perform assorted control functions associated with a connected external device, in response to the external device being connected to the interface unit 160.

The memory 170 is typically implemented to store data to support various functions or features of the mobile terminal 100. For instance, the memory 170 may be configured to store application programs executed in the mobile terminal 100, data or instructions for operations of the mobile terminal 100, and the like. Some of these application programs may be downloaded from an external server via wireless communication. Other application programs may be installed within the mobile terminal 100 at time of manufacturing or shipping, which is typically the case for basic functions of the mobile terminal 100 (for example, receiving a call, placing a call, receiving a message, sending a message, and the like). Application programs may be stored in the memory 170, installed in the mobile terminal 100, and executed by the controller 180 to perform an operation (or function) for the mobile terminal 100.

The controller 180 typically functions to control an overall operation of the mobile terminal 100, in addition to the operations associated with the application programs. The controller 180 may provide or process information or functions appropriate for a user by processing signals, data, information and the like, which are input or output by the aforementioned various components, or activating application programs stored in the memory 170.

Also, the controller 180 may control at least some of the components illustrated in FIG. 1A, to execute an application program that have been stored in the memory 170. In addition, the controller 180 may control at least two of those components included in the mobile terminal 100 to activate the application program.

The power supply unit 190 may be configured to receive external power or provide internal power in order to supply appropriate power required for operating elements and components included in the mobile terminal 100. The power supply unit 190 may include a battery, and the battery may be configured to be embedded in the terminal body, or configured to be detachable from the terminal body.

At least part of the components may cooperatively operate to implement an operation, a control or a control method of a mobile terminal according to various embodiments disclosed herein. Also, the operation, the control or the control method of the mobile terminal may be implemented on the mobile terminal by an activation of at least one application program stored in the memory 170.

Hereinafter, description will be given in more detail of the aforementioned components with reference to FIG. 1A, prior to describing various embodiments implemented through the mobile terminal 100. First, regarding the wireless communication unit 110, the broadcast receiving module 111 is typically configured to receive a broadcast signal and/or broadcast associated information from an external broadcast managing entity via a broadcast channel. The broadcast channel may include a satellite channel, a terrestrial channel, or both. In some embodiments, two or more broadcast receiving modules may be utilized to facilitate simultaneous reception of two or more broadcast channels, or to support switching among broadcast channels.

The mobile communication module 112 can transmit and/or receive wireless signals to and from one or more network entities. Typical examples of a network entity include a base station, an external mobile terminal, a server, and the like. Such network entities form part of a mobile communication network, which is constructed according to technical standards or communication methods for mobile communications (for example, Global System for Mobile Communication (GSM), Code Division Multi Access (CDMA), CDMA2000 (Code Division Multi Access 2000), EV-DO (Enhanced Voice-Data Optimized or Enhanced Voice-Data Only), Wideband CDMA (WCDMA), High Speed Downlink Packet access (HSDPA), HSUPA (High Speed Uplink Packet Access), Long Term Evolution (LTE), LTE-A (Long Term Evolution-Advanced), and the like).

The wireless signal may include various types of data depending on a voice call signal, a video call signal, or a text/multimedia message transmission/reception. The wireless Internet module 113 refers to a module for wireless Internet access. This module may be internally or externally coupled to the mobile terminal 100. The wireless Internet module 113 may transmit and/or receive wireless signals via communication networks according to wireless Internet technologies.

Examples of such wireless Internet access include Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), High Speed Uplink Packet Access (HSUPA), Long Term Evolution (LTE), LTE-advanced (LTE-A) and the like. The wireless Internet module 113 may transmit/receive data according to one or more of such wireless Internet technologies, and other Internet technologies as well.

When the wireless Internet access is implemented according to, for example, WiBro, HSDPA, HSUPA, GSM, CDMA, WCDMA, LTE, LTE-A and the like, as part of a mobile communication network, the wireless Internet module 113 performs such wireless Internet access. As such, the Internet module 113 may cooperate with, or function as, the mobile communication module 112.

The short-range communication module 114 is configured to facilitate short-range communications. Suitable technologies for implementing such short-range communications include BLUETOOTH™, Radio Frequency IDentification (RFID), Infrared Data Association (IrDA), Ultra-WideBand (UWB), ZigBee, Near Field Communication (NFC), Wireless-Fidelity (Wi-Fi), Wi-Fi Direct, Wireless USB (Wireless Universal Serial Bus), and the like. The short-range communication module 114 in general supports wireless communications between the mobile terminal 100 and a wireless communication system, communications between the mobile terminal 100 and another mobile terminal 100, or communications between the mobile terminal and a network where another mobile terminal 100 (or an external server) is located, via wireless area networks. One example of the wireless area networks is a wireless personal area network.

Here, another mobile terminal (which may be configured similarly to mobile terminal 100) may be a wearable device, for example, a smart watch, a smart glass or a head mounted display (HMD), which is able to exchange data with the mobile terminal 100 (or otherwise cooperate with the mobile terminal 100). The short-range communication module 114 may sense or recognize the wearable device, and permit communication between the wearable device and the mobile terminal 100. In addition, when the sensed wearable device is a device which is authenticated to communicate with the mobile terminal 100, the controller 180, for example, may cause transmission of at least part of data processed in the mobile terminal 100 to the wearable device via the short-range communication module 114. Hence, a user of the wearable device may use the data processed in the mobile terminal 100 on the wearable device. For example, when a call is received in the mobile terminal 100, the user may answer the call using the wearable device. Also, when a message is received in the mobile terminal 100, the user can check the received message using the wearable device.

The location information module 115 is generally configured to detect, calculate, derive or otherwise identify a position (or current position) of the mobile terminal. As an example, the location information module 115 includes a Global Position System (GPS) module, a Wi-Fi module, or both. For example, when the mobile terminal uses a GPS module, a position of the mobile terminal may be acquired using a signal sent from a GPS satellite. As another example, when the mobile terminal uses the Wi-Fi module, a position of the mobile terminal can be acquired based on information related to a wireless access point (AP) which transmits or receives a wireless signal to or from the Wi-Fi module. If desired, the location information module 115 may alternatively or additionally function with any of the other modules of the wireless communication unit 110 to obtain data related to the position of the mobile terminal. The location information module 115 is a module used for acquiring the position (or the current position) and may not be limited to a module for directly calculating or acquiring the position of the mobile terminal.

Next, the input unit 120 is for inputting image information (or signal), audio information (or signal), data, or information input from a user. For inputting image information, the mobile terminal 100 may be provided with a plurality of cameras 121. Such cameras 121 may process image frames of still pictures or video obtained by image sensors in a video or image capture mode. The processed image frames can be displayed on the display unit 151 or stored in memory 170. Meanwhile, the cameras 121 may be arranged in a matrix configuration to permit a plurality of images having various angles or focal points to be input to the mobile terminal 100. Also, the cameras 121 may be located in a stereoscopic arrangement to acquire left and right images for implementing a stereoscopic image.

The microphone 122 processes an external audio signal into electric audio (sound) data. The processed audio data can be processed in various manners according to a function being executed in the mobile terminal 100. If desired, the microphone 122 may include assorted noise removing algorithms to remove unwanted noise generated in the course of receiving the external audio signal.

The user input unit 123 is a component that permits input by a user. Such user input may enable the controller 180 to control operation of the mobile terminal 100. The user input unit 123 may include one or more of a mechanical input element (for example, a mechanical key, a button located on a front and/or rear surface or a side surface of the mobile terminal 100, a dome switch, a jog wheel, a jog switch, and the like), or a touch-sensitive input element, among others. As one example, the touch-sensitive input element may be a virtual key, a soft key or a visual key, which is displayed on a touch screen through software processing, or a touch key which is located on the mobile terminal at a location that is other than the touch screen. On the other hand, the virtual key or the visual key may be displayed on the touch screen in various shapes, for example, graphic, text, icon, video, or a combination thereof.

The sensing unit 140 is generally configured to sense one or more of internal information of the mobile terminal, surrounding environment information of the mobile terminal, user information, or the like, and generate a corresponding sensing signal. The controller 180 generally cooperates with the sending unit 140 to control operations of the mobile terminal 100 or execute data processing, a function or an operation associated with an application program installed in the mobile terminal based on the sensing signal. The sensing unit 140 may be implemented using any of a variety of sensors, some of which will now be described in more detail.

The proximity sensor 141 refers to a sensor to sense presence or absence of an object approaching a surface, or an object located near a surface, by using an electromagnetic field, infrared rays, or the like without a mechanical contact. The proximity sensor 141 may be arranged at an inner region of the mobile terminal covered by the touch screen, or near the touch screen.

The proximity sensor 141, for example, may include any of a transmissive type photoelectric sensor, a direct reflective type photoelectric sensor, a mirror reflective type photoelectric sensor, a high-frequency oscillation proximity sensor, a capacitance type proximity sensor, a magnetic type proximity sensor, an infrared rays proximity sensor, and the like. When the touch screen is implemented as a capacitance type, the proximity sensor 141 can sense proximity of a pointer relative to the touch screen by changes of an electromagnetic field, which is responsive to an approach of an object with conductivity. In this case, the touch screen (touch sensor) may also be categorized as a proximity sensor.

The term “proximity touch” will often be referred to herein to denote the scenario in which a pointer is positioned to be proximate to the touch screen without contacting the touch screen. The term “contact touch” will often be referred to herein to denote the scenario in which a pointer makes physical contact with the touch screen. For the position corresponding to the proximity touch of the pointer relative to the touch screen, such position will correspond to a position where the pointer is perpendicular to the touch screen. The proximity sensor 141 may sense proximity touch, and proximity touch patterns (for example, distance, direction, speed, time, position, moving status, and the like). In general, controller 180 processes data corresponding to proximity touches and proximity touch patterns sensed by the proximity sensor 141, and cause output of visual information on the touch screen. In addition, the controller 180 can control the mobile terminal 100 to execute different operations or process different data (or information) according to whether a touch with respect to a point on the touch screen is either a proximity touch or a contact touch.

A touch sensor senses a touch (or a touch input) applied to the touch screen (or the display unit 151) using any of a variety of touch methods. Examples of such touch methods include a resistive type, a capacitive type, an infrared type, and a magnetic field type, among others.

As one example, the touch sensor may be configured to convert changes of pressure applied to a specific part of the display unit 151, or convert capacitance occurring at a specific part of the display unit 151, into electric input signals. The touch sensor may also be configured to sense not only a touched position and a touched area, but also touch pressure and/or touch capacitance. A touch object is generally used to apply a touch input to the touch sensor. Examples of typical touch objects include a finger, a touch pen, a stylus pen, a pointer, or the like.

When a touch input is sensed by a touch sensor, corresponding signals may be transmitted to a touch controller. The touch controller may process the received signals, and then transmit corresponding data to the controller 180. Accordingly, the controller 180 may sense which region of the display unit 151 has been touched. Here, the touch controller may be a component separate from the controller 180, the controller 180, and combinations thereof.

Meanwhile, the controller 180 may execute the same or different controls according to a type of touch object that touches the touch screen or a touch key provided in addition to the touch screen. Whether to execute the same or different control according to the object which provides a touch input may be decided based on a current operating state of the mobile terminal 100 or a currently executed application program, for example.

The touch sensor and the proximity sensor may be implemented individually, or in combination, to sense various types of touches. Such touches include a short (or tap) touch, a long touch, a multi-touch, a drag touch, a flick touch, a pinch-in touch, a pinch-out touch, a swipe touch, a hovering touch, and the like.

If desired, an ultrasonic sensor may be implemented to recognize location information relating to a touch object using ultrasonic waves. The controller 180, for example, may calculate a position of a wave generation source based on information sensed by an illumination sensor and a plurality of ultrasonic sensors. Since light is much faster than ultrasonic waves, the time for which the light reaches the optical sensor is much shorter than the time for which the ultrasonic wave reaches the ultrasonic sensor. The position of the wave generation source may be calculated using this fact. For instance, the position of the wave generation source may be calculated using the time difference from the time that the ultrasonic wave reaches the sensor based on the light as a reference signal.

The camera 121, which has been depicted as a component of the input unit 120, typically includes at least one a camera sensor (CCD, CMOS etc.), a photo sensor (or image sensors), and a laser sensor.

Implementing the camera 121 with a laser sensor may allow detection of a touch of a physical object with respect to a 3D stereoscopic image. The photo sensor may be laminated on, or overlapped with, the display device. The photo sensor may be configured to scan movement of the physical object in proximity to the touch screen. In more detail, the photo sensor may include photo diodes and transistors (TRs) at rows and columns to scan content received at the photo sensor using an electrical signal which changes according to the quantity of applied light. Namely, the photo sensor may calculate the coordinates of the physical object according to variation of light to thus obtain location information of the physical object.

The display unit 151 is generally configured to output information processed in the mobile terminal 100. For example, the display unit 151 may display execution screen information of an application program executing at the mobile terminal 100 or user interface (UI) and graphic user interface (GUI) information in response to the execution screen information.

Also, the display unit 151 may be implemented as a stereoscopic display unit for displaying stereoscopic images. A typical stereoscopic display unit 151 may employ a stereoscopic display scheme such as a stereoscopic scheme (a glass scheme), an auto-stereoscopic scheme (glassless scheme), a projection scheme (holographic scheme), or the like.

The audio output module 152 may receive audio data from the wireless communication unit 110 or output audio data stored in the memory 170 during modes such as a signal reception mode, a call mode, a record mode, a voice recognition mode, a broadcast reception mode, and the like. The audio output module 152 can provide audible output related to a particular function (e.g., a call signal reception sound, a message reception sound, etc.) performed by the mobile terminal 100. The audio output module 152 may also be implemented as a receiver, a speaker, a buzzer, or the like.

A haptic module 153 can be configured to generate various tactile effects that a user feels, perceives, or otherwise experiences. A typical example of a tactile effect generated by the haptic module 153 is vibration. The strength, pattern and the like of the vibration generated by the haptic module 153 may be controlled by user selection or setting by the controller 180. For example, the haptic module 153 may output different vibrations in a combining manner or a sequential manner.

Besides vibration, the haptic module 153 can generate various other tactile effects, including an effect by stimulation such as a pin arrangement vertically moving to contact skin, a spray force or suction force of air through a jet orifice or a suction opening, a touch to the skin, a contact of an electrode, electrostatic force, an effect by reproducing the sense of cold and warmth using an element that can absorb or generate heat, and the like.

The haptic module 153 can also be implemented to allow the user to feel a tactile effect through a muscle sensation such as the user's fingers or arm, as well as transferring the tactile effect through direct contact. Two or more haptic modules 153 may be provided according to the particular configuration of the mobile terminal 100.

An optical output module 154 can output a signal for indicating an event generation using light of a light source. Examples of events generated in the mobile terminal 100 may include message reception, call signal reception, a missed call, an alarm, a schedule notice, an email reception, information reception through an application, and the like.

A signal output by the optical output module 154 may be implemented in such a manner that the mobile terminal emits monochromatic light or light with a plurality of colors. The signal output may be terminated as the mobile terminal senses that a user has checked the generated event, for example.

The interface unit 160 serves as an interface for external devices to be connected with the mobile terminal 100. For example, the interface unit 160 can receive data transmitted from an external device, receive power to transfer to elements and components within the mobile terminal 100, or transmit internal data of the mobile terminal 100 to such external device. The interface unit 160 may include wired or wireless headset ports, external power supply ports, wired or wireless data ports, memory card ports, ports for connecting a device having an identification module, audio input/output (I/O) ports, video I/O ports, earphone ports, or the like.

The identification module may be a chip that stores various information for authenticating authority of using the mobile terminal 100 and may include a user identity module (UIM), a subscriber identity module (SIM), a universal subscriber identity module (USIM), and the like. In addition, the device having the identification module (also referred to herein as an “identifying device”) may take the form of a smart card. Accordingly, the identifying device can be connected with the terminal 100 via the interface unit 160.

When the mobile terminal 100 is connected with an external cradle, the interface unit 160 can serve as a passage to allow power from the cradle to be supplied to the mobile terminal 100 or may serve as a passage to allow various command signals input by the user from the cradle to be transferred to the mobile terminal therethrough. Various command signals or power input from the cradle may operate as signals for recognizing that the mobile terminal is properly mounted on the cradle.

The memory 170 can store programs to support operations of the controller 180 and store input/output data (for example, phonebook, messages, still images, videos, etc.). The memory 170 may store data related to various patterns of vibrations and audio which are output in response to touch inputs on the touch screen.

The memory 170 may include one or more types of storage mediums including a flash memory type, a hard disk type, a solid state disk (SSD) type, a silicon disk drive (SDD) type, a multimedia card micro type, a card-type memory (e.g., SD or DX memory, etc.), a Random Access Memory (RAM), a Static Random Access Memory (SRAM), a Read-Only Memory (ROM), an Electrically Erasable Programmable Read-Only Memory (EEPROM), a Programmable Read-Only memory (PROM), a magnetic memory, a magnetic disk, an optical disk, and the like. The mobile terminal 100 may also be operated in relation to a network storage device that performs the storage function of the memory 170 over a network, such as the Internet.

The controller 180 may typically control operations relating to application programs and the general operations of the mobile terminal 100. For example, the controller 180 may set or release a lock state for restricting a user from inputting a control command with respect to applications when a status of the mobile terminal meets a preset condition.

The controller 180 can also perform the controlling and processing associated with voice calls, data communications, video calls, and the like, or perform pattern recognition processing to recognize a handwriting input or a picture drawing input performed on the touch screen as characters or images, respectively. In addition, the controller 180 can control one or a combination of those components in order to implement various exemplary embodiments disclosed herein.

The power supply unit 190 receives external power or provides internal power and supply the appropriate power required for operating respective elements and components included in the wearable device 100 under the control of the controller 180. The power supply unit 190 may include a battery, which is typically rechargeable or be detachably coupled to the terminal body for charging.

The power supply unit 190 may include a connection port. The connection port may be configured as one example of the interface unit 160 to which an external charger for supplying power to recharge the battery is electrically connected. As another example, the power supply unit 190 may be configured to recharge the battery in a wireless manner without use of the connection port.

In this example, the power supply unit 190 can receive power, transferred from an external wireless power transmitter, using at least one of an inductive coupling method which is based on magnetic induction or a magnetic resonance coupling method which is based on electromagnetic resonance. Various embodiments described herein may be implemented in a computer-readable medium, a machine-readable medium, or similar medium using, for example, software, hardware, or any combination thereof.

FIG. 1B is a view of a watch-type terminal according to one embodiment, viewed from one direction. Referring to FIG. 1B, a watch-type terminal 100 includes a main body 101 having a display unit 151, and a band 102 connected to the main body 101 and configured to be worn on a wrist.

The main body 101 includes a case which defines appearance. As illustrated, the case may include a first case 101a and a second case 101b cooperatively defining an inner space for accommodating various electronic components. However, the present invention is not limited to this, and one case may be configured to define the inner space, thereby implementing a terminal 100 with a uni-body.

The watch-type terminal 100 can perform wireless communication, and an antenna for the wireless communication can be installed in the main body 101. On the other hand, the antenna may extend its function using the case. For example, a case including a conductive material may be electrically connected to the antenna to extend a ground area or a radiation area.

The display unit 151 may be disposed on a front surface of the main body 101 to output information, and a touch sensor may be provided on the display unit 151 to implement a touch screen. As illustrated, a window 151a of the display unit 151 may be mounted on a first case 101a to form the front surface of the terminal body together with the first case 101a.

The main body 101 may include an audio output unit 152, a camera 121, a microphone 122, a user input unit 123, and the like. When the display unit 151 is implemented as the touch screen, the display unit 351 may function as a user input unit 123, so that the main body 101 may not have a separate key.

The band 102 may be worn on the wrist so as to surround the wrist, and may be formed of a flexible material for easy wearing. As an example, the band 102 may be formed of leather, rubber, silicone, synthetic resin, or the like. The band 102 may be detachably attached to the main body 101, and may be configured to be replaceable with various types of bands according to the user's preference.

On the other hand, the band 102 may be used to extend the performance of the antenna. For example, the band may include a ground extending portion (not illustrated) that is electrically connected to the antenna and extends a ground region.

The band 102 may be provided with a fastener 102a. The fastener 102a may be embodied by a buckle type, a snap-fit hook structure, a Velcro® type, or the like, and include a flexible section or material. The drawing illustrates an example that the fastener 102a is implemented using a buckle.

FIG. 1C is a conceptual view of a watch-type terminal according to one embodiment of the present invention, viewed from one direction. The watch-type terminal 100 according to the present invention includes a sensor module for measuring a biological signal. In the watch-type terminal 100 according to this embodiment, a rear cover 101c is provided on a surface facing the display unit 151. The rear cover 101c forms an inner space together with the second case 101b.

A receiving portion 301 for receiving a first sensor module 310 is formed on the rear cover 101c. The receiving portion 301 is formed to protrude from an outer surface of the rear cover 101c and provided with a window having a light-transmissive area in which light emitted from a first sensor unit 310 and reflected by a user's body is received. The receiving portion 301 may receive therein a user identity module (UIM), a subscriber identity module (SIM), a universal subscriber identity module (USIM), and the like.

The first sensing module 310 may be closely adhered to one area of the user's body by the receiving portion 301 protruded from the second case 101b, which may result in minimizing a leakage of emitted light.

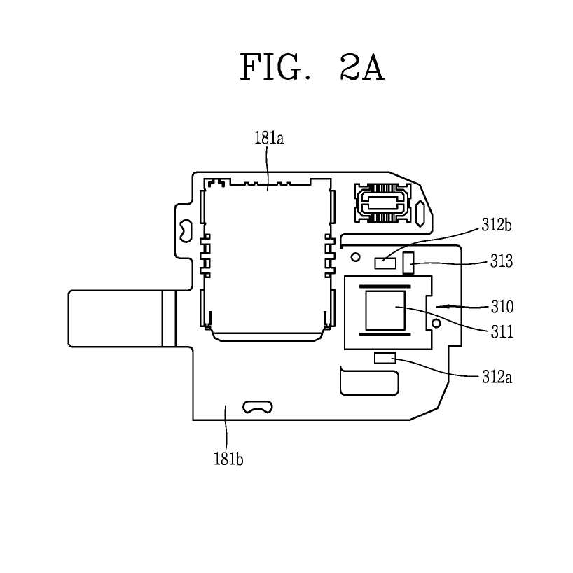

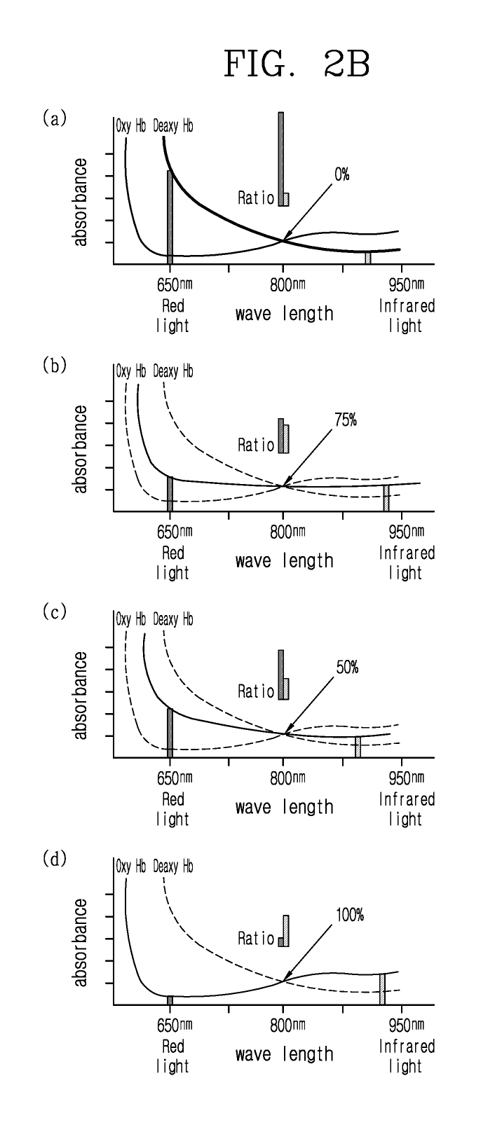

FIG. 2A is a conceptual view illustrating a configuration and an arrangement structure of a sensing module. In FIG. 2A, a chip 181a and the first sensor unit 310 are provided on a circuit board 181b. The first sensor unit 310 includes a light-receiving sensor 311, a first light-emitting element 312a, and a second light-emitting element 312b. The first and second light-emitting elements 312a and 312b are disposed on the circuit board 181b with the light-receiving sensor 311 interposed therebetween. The light-receiving sensor 311 and the first and second light-emitting elements 312a and 312b are independently fixed to the circuit board 181b and are spaced apart from each other by a preset distance. Also, an IR sensor 313 is disposed adjacent to the second light-emitting element 312b. The light-emitting element may be an LED device that outputs green light.

The first and second light-emitting elements 312a and 312b output green light. The green light output from the first and second light-emitting elements 312a and 312b is reflected by a skin and is received by the light-receiving sensor 311.

Transmittance is decreased when light has a short wavelength and increased when light has a long wavelength. In order to measure a biological signal (a heartbeat change) as a PPG sensor, the output light should reach a skin depth where blood vessels are located, to measure a change in a blood flow. However, when light reaches beyond the skin depth where the blood vessels are located, it may be absorbed into tissues or bones. In general, depth from a wrist to a blood vessel is deeper than that from a finger to a blood vessel, and thus the transmittance of green light is suitable for reaching the blood vessel.

The sensing unit according to this embodiment includes a red light-emitting element and an IR element for measuring an oxygen saturation. The red light and the IR have high absorption rates of hemoglobin (Hb) and oxygen hemoglobin (HbO2), and the absorption rates are different from each other. Accordingly, the oxygen saturation is calculated through a ratio of the absorption rate of oxygen hemoglobin (HbO2) to the sum of the absorption rate of oxygen hemoglobin (HbO2) and the absorption rate of hemoglobin (Hb).

Since the oxygen hemoglobin (HbO2) and the hemoglobin (Hb) have different absorbances of red light and IR light, graphs of the ratios thereof are also formed differently.

FIG. 2B is a graph illustrating a light absorption rate of hemoglobin (Hb) and oxygen hemoglobin (HbO2) according to a wavelength of light. (a) of FIG. 2B is a graph showing an amount of light absorbed when oxygen hemoglobin (HbO2) does not exist in blood (dead person). In this case, since there is no absorbed light of the oxygen hemoglobin (HbO2), the oxygen saturation is 0%.

Referring to (d) of FIG. 2B, when all hemoglobin is bound to oxygen, a graph showing the oxygen saturation is formed substantially the same as a graph showing a light absorbance according to the wavelength of oxygen saturation (HbO2). This indicates a state in which all of the oxygen and the hemoglobin are bound together and thus the oxygen can be delivered to the full body. (b) of FIG. 2B and (c) of FIG. 2B are graphs showing different oxygen saturations.

However, in order to calculate the oxygen saturation according to the ratio of the hemoglobin (Hb) and the oxygen hemoglobin (HbO2), the IR sensor and the red light-emitting element should be spaced apart from each other by about 6 mm to 8 mm. The light-emitting elements and the light-receiving sensor of the sensing unit 310 according to this embodiment are not formed as one module but arranged on the circuit board. Accordingly, the watch-type terminal 100 may be provided with a light-emitting element 312 and a light-receiving sensor 311 which are arranged to maintain a sufficient distance therebetween.

Hereinafter, the arrangement structure of the light-receiving sensor 311 and the light-emitting element 312 included in the sensing unit 310 will be described.

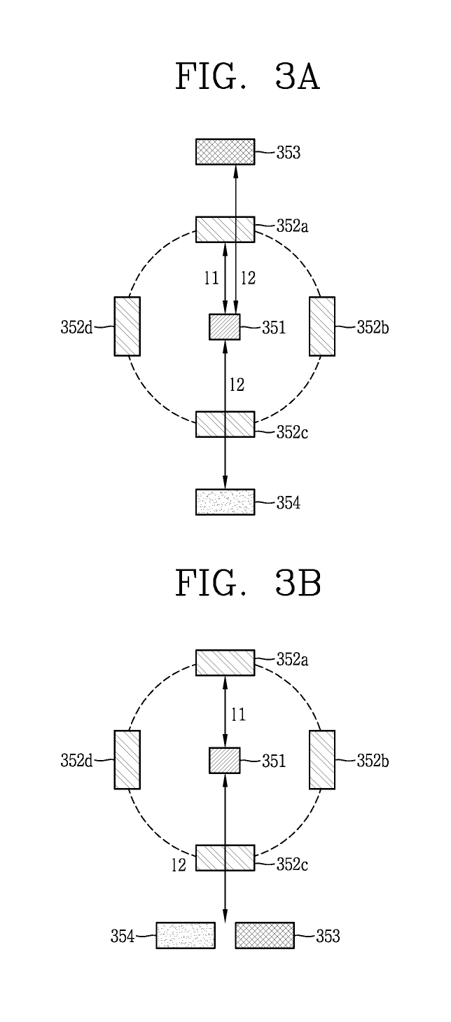

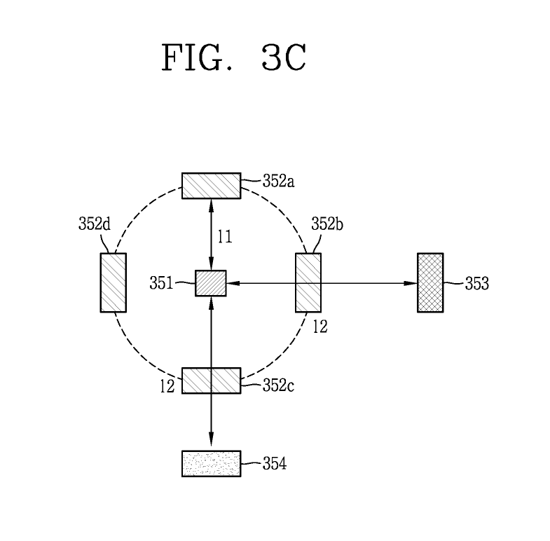

FIGS. 3A to 3C are conceptual views illustrating a sensor unit for outputting red light for measuring oxygen saturation. A sensor unit in FIG. 3A includes a first light-receiving sensor 351, first to fourth green light-emitting elements 352a, 352b, 352c, and 352d, an IR sensor 353, and a red light-emitting element 354. The first to fourth green light-emitting elements 352a, 352b, 352c, and 352d may be LED devices that output green light. The green light output from the first to fourth green light-emitting elements 352a, 352b, 352c, and 352d is reflected by a skin and is received by the first light-receiving sensor 351.

Transmittance is decreased when light has a short wavelength and increased when light has a long wavelength. In order to measure a biological signal (heartbeat change) as a PPG sensor, the output light should reach a skin depth where blood vessels are located, to measure a change in a blood flow. However, when light reaches beyond the skin depth where the blood vessels are located, it may be absorbed into tissues or bones. In general, depth from a wrist to a blood vessel is deeper than that from a finger to the blood vessel, and thus the transmittance of green light is suitable for reaching the blood vessel.

The first to fourth green light-emitting elements 352a, 352b, 352c, and 352d are disposed to be spaced apart from one another by a first length 11 with respect to the first light-receiving sensor 351. On the other hand, the IR sensor 353 is disposed in parallel (side by side) to the first green light-emitting element 352a and is spaced apart from the first light-receiving sensor 351 by a second length 12 longer than the first length 11.

The red light-emitting element 354 is disposed in parallel to the third green light-emitting element 352c and is spaced apart from the first light-receiving sensor 351 by the second length 12. According to this embodiment, the IR sensor 353 and the red light-emitting element 354 may be disposed at the farthest distance from each other. For example, the second length 12 may range from about 6 mm to about 8 mm.

Referring to FIG. 3B, the IR sensor 353 and the red light-emitting element 354 may be disposed adjacent to each other. According to this embodiment, the IR sensor 353 and the red light-emitting element 354 are disposed in series (side by side) to each other and are spaced apart from the first light-receiving sensor 351 by the second length 12. The IR sensor 383 and the red light-emitting element 354 may be disposed adjacent to one of the plurality of green light-emitting elements.

Referring to FIG. 3C, the IR sensor 353 and the red light-emitting element 354 are spaced apart from the first light-receiving sensor 351 by the second length 12, respectively. The IR sensor 353 may be adjacent to the second green light-emitting element 352b and the red light-emitting element 354 may be disposed adjacent to the third green light-emitting element 352c.

According to these embodiments, output intensity of the green light of the green light-emitting element may be adjusted to fit the user's skin so as to measure a biological signal, and the oxygen saturation may be measured using the red light. Also, the IR sensor may be used to detect whether or not the watch-type terminal is worn.

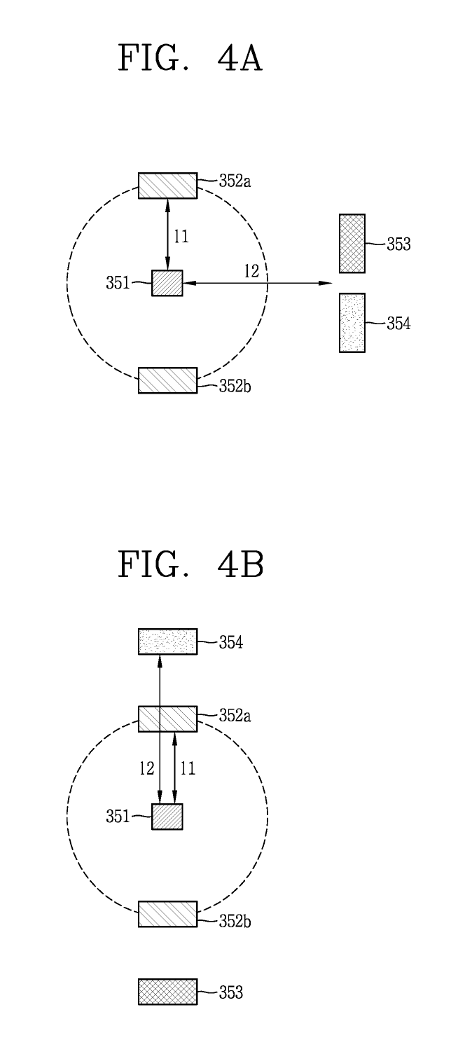

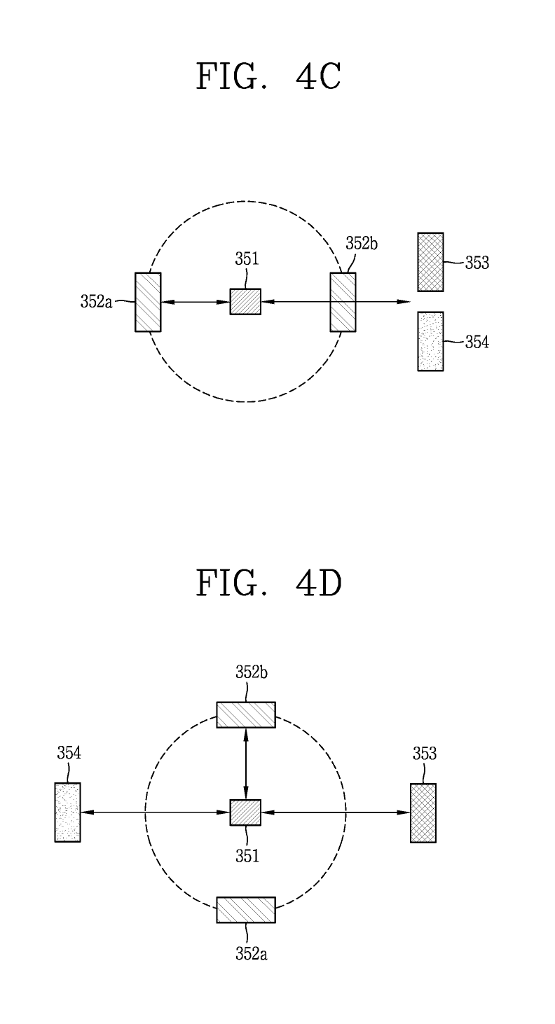

FIGS. 4A to 4D are conceptual views illustrating a sensor unit for outputting red light for measuring an oxygen saturation according to another embodiment. Referring to FIG. 4A, the sensor unit according to this embodiment includes the first and second green light-emitting elements 352a and 352b, the IR sensor 353, and the red light-emitting element 354. In other words, the sensor unit according to this embodiment has the same configuration as that illustrated in FIGS. 3A to 3C, except for including only two green light-emitting elements. Thus, the same reference numerals are used and a redundant description will be omitted.

The first and second green light-emitting elements 352a and 352b are disposed along a first direction d1 with the light-receiving sensor 351 interposed therebetween. The first and second green light-emitting elements 352a and 52b are spaced apart from the light-receiving sensor 351 by a first length 11, respectively.

The IR sensor 353 and the red light-emitting element 354 are disposed adjacent to each other and are spaced apart from the light-receiving sensor 351 by a second length 12. The IR sensor 353 and the red light-emitting element 354 are arranged apart from the light-receiving sensor 351 along a second direction d2 intersecting with the first direction d1.

Referring to FIG. 4B, the first and second green light-emitting elements 352a and 352b, the red light-emitting element 354, the IR sensor 353 and the light-receiving sensor 351 are arranged in the first direction d1. The IR sensor 353 and the red light-emitting element 354 are spaced apart from the light-receiving sensor 351 by the second length 12, respectively. The second green light-emitting element 352b is disposed between the IR sensor 353 and the light-receiving sensor 351 and the first green light-emitting element 352a is disposed between the light-receiving sensor 351 and the red light-emitting element 354.

Referring to FIG. 4C, the red light-emitting element 354 and the IR sensor 353 are disposed adjacent to each other and spaced apart from the light-receiving sensor 351 by the second length 12. The second green light-emitting element 352b is disposed between the light-receiving sensor 351 and the red light-emitting element 354 and the IR sensor 353. The first green light-emitting element 352a is arranged to correspond to the first green light-emitting element 352a with respect to the light-receiving sensor 351.

Referring to FIG. 4D, the first and second green light-emitting elements 352a and 352b, the red light-emitting element 354, and the IR sensor 353 are disposed in all directions, with respect to the light-receiving sensor 351. Even in this case, the red light-emitting element 354 and the IR sensor 353 are spaced apart from the light-receiving sensor 351 by the second length 12, respectively, and the first and second green light-emitting elements 352a and 352b are spaced apart from the light-receiving sensor 351 by the first length 11, respectively.

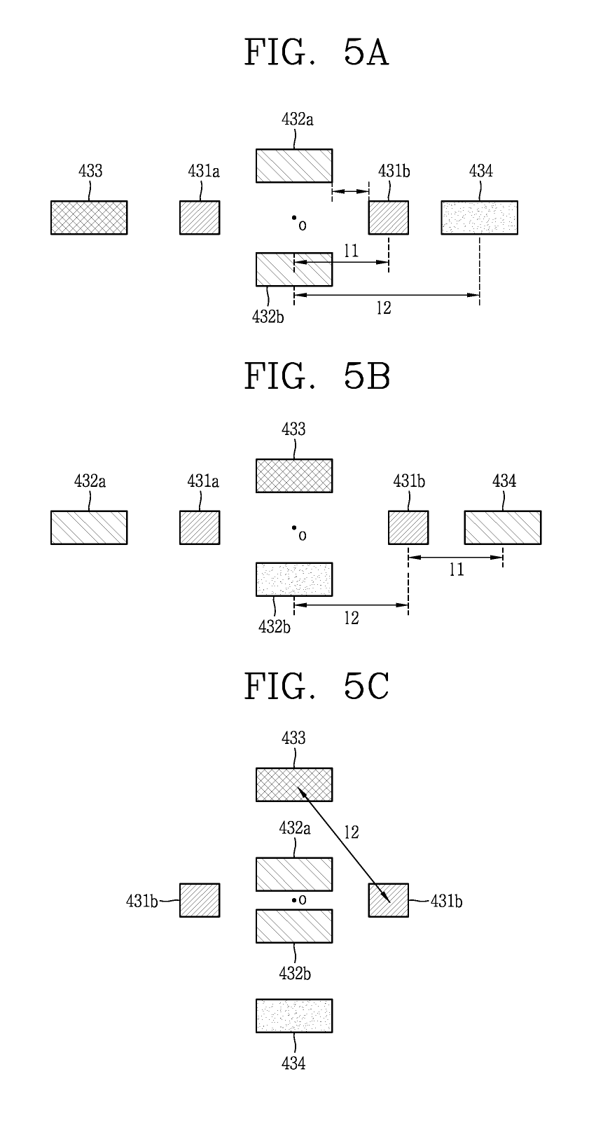

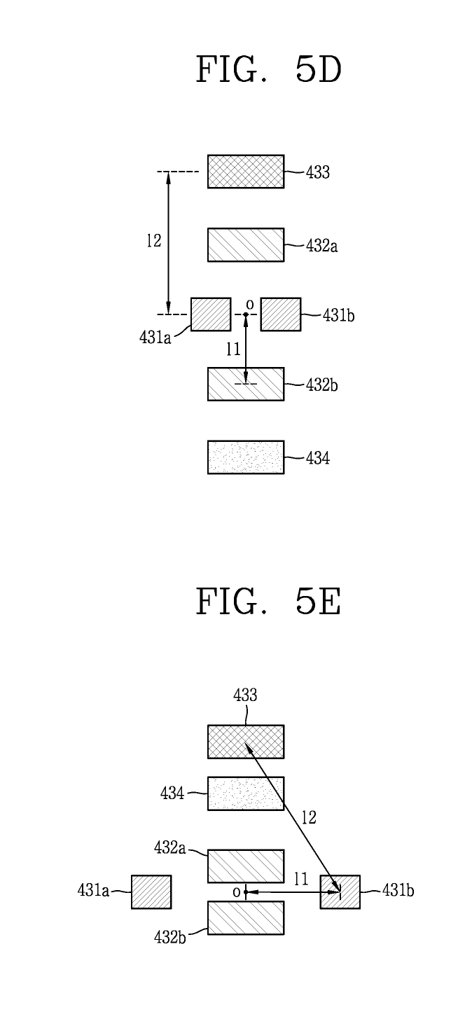

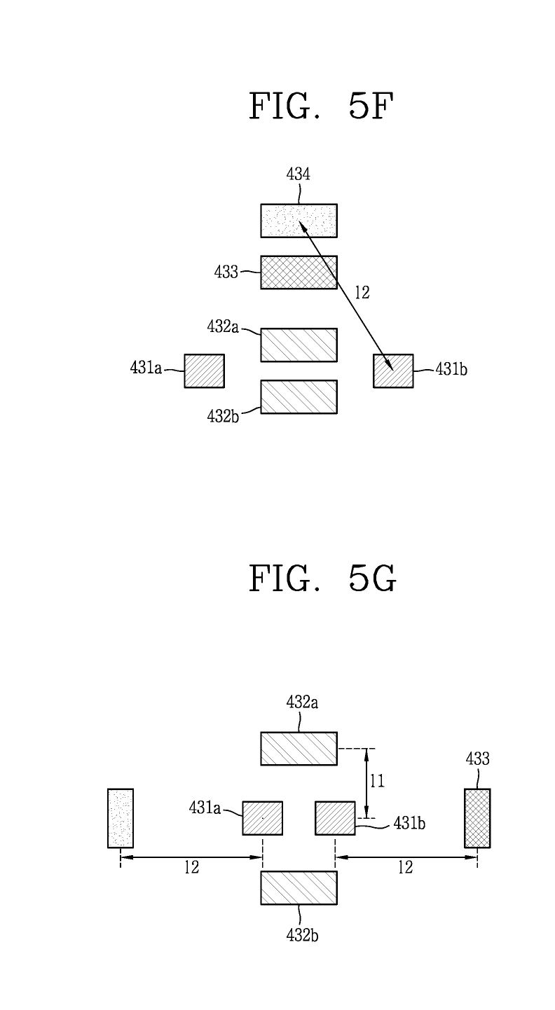

FIGS. 5A to 5G are conceptual views illustrating a sensing unit which includes two light-receiving sensors and is capable of measuring oxygen saturation. Referring to FIG. 5A, first and second light-receiving sensors 431a and 431b are arranged along a first direction with respect to a virtual center O. The first and second light-receiving sensors 431a and 431b are spaced apart from the center O by the first length 11, respectively.

The IR sensor 433 and the red light-emitting element 434 are arranged along the first direction and spaced apart from the center O by the second length 12, respectively. The first and second green light-emitting elements 432a and 432b are arranged along a second direction that intersects with the first direction, and spaced apart from the first and second light-emitting elements 431a and 431b by the first length 11, respectively.

Referring to FIG. 5B, the first and second green light-emitting elements 432a and 432b and the first and second light-receiving sensors 431a and 431b are arranged along the first direction. The first and second light-receiving sensors 431a and 431b are spaced apart from the virtual center O by the second length 12, respectively. The first and second green light-emitting elements 432a and 432b are spaced apart from the first and second light-receiving sensors 431a and 431b by the first length 11, and disposed outside the first and second light-receiving sensors 431a and 431b, respectively. The IR sensor 433 and the red light-emitting element 434 are arranged along the second direction intersecting with the first direction.

Referring to FIG. 5C, the first and second green light-emitting elements 432a and 432b are disposed adjacent to each other based on the virtual center O. The red light-emitting element 434, the IR sensor 433 and the first and second light-receiving sensors 431a and 431b are arranged in all directions with respect to the virtual center O. The first and second green light-emitting elements 432a and 432b, the IR sensor 433, and the red light-emitting element 434 are arranged in one direction. The first and second light-receiving sensors 431a and 431b are disposed closer to the first and second green light-emitting elements 432a and 432b, respectively.

Referring to FIG. 5D, the first and second green light-emitting elements 432a and 432b and the first and second light-receiving sensors 431a and 431b are disposed in all directions with respect to the virtual center O. The IR sensor 433 and the red light-emitting element 434 are spaced apart from the virtual center O by the second length 12, respectively, and arranged along a direction that the first and second green light-emitting elements 432a and 432b are arranged.

Referring to FIG. 5E, the first and second green light-emitting elements 432a and 432b, the IR sensor 434, and the red light-emitting element 433 are arranged in one direction with respect to the center O. The first and second light-receiving sensors 431a and 431b are arranged in a direction intersecting with the one direction with respect to the center O. The first and second light-receiving sensors 431a and 431b are arranged to be close to the first and second green light-emitting elements 432a and 432b and relatively far from the IR sensor 434 and the red light-emitting element 433. The first and second light-receiving sensors 431a and 431b and the IR sensor 434 are preferably spaced apart from each other by the second length 12, respectively.

As illustrated in FIG. 5F, the positions of the IR sensor 434 and the red light-emitting element 433 may be changed. Referring to FIG. 5G, the first and second light-receiving sensors 431a and 431b are disposed adjacent to each other and the IR sensor 434 and the red light-emitting element 433 are disposed adjacent to the first and second light-receiving sensors 431a and 431b, respectively. The first and second green light-emitting elements 432a and 432b are arranged in a direction intersecting with a direction in which the IR sensor 434, the red light-emitting element 433 and the first and second light-receiving sensors 431a and 431b are arranged.

According to the present invention, since the light-receiving sensors, the green light-emitting elements, the red light-emitting element, and the IR sensor can be disposed separately, not as one module, the distances between the red light-emitting element and the light-receiving sensors and between the IR sensor and the light-receiving sensors can be secured. Therefore, the oxygen saturation can be measured more accurately.

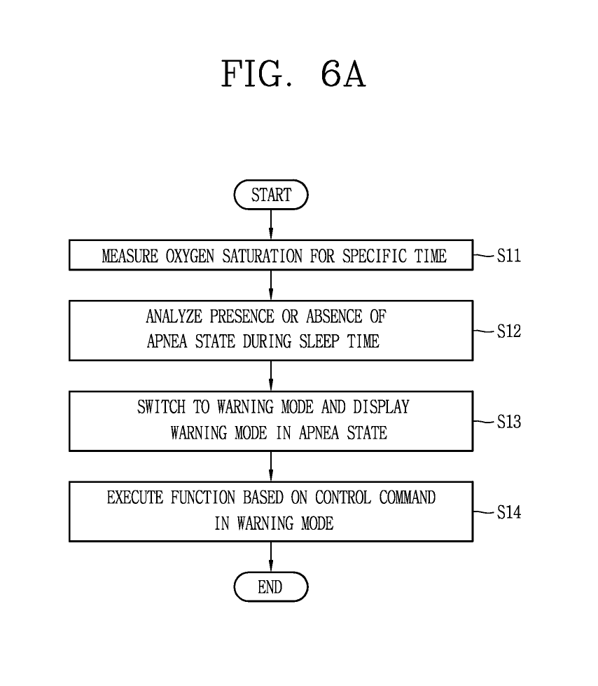

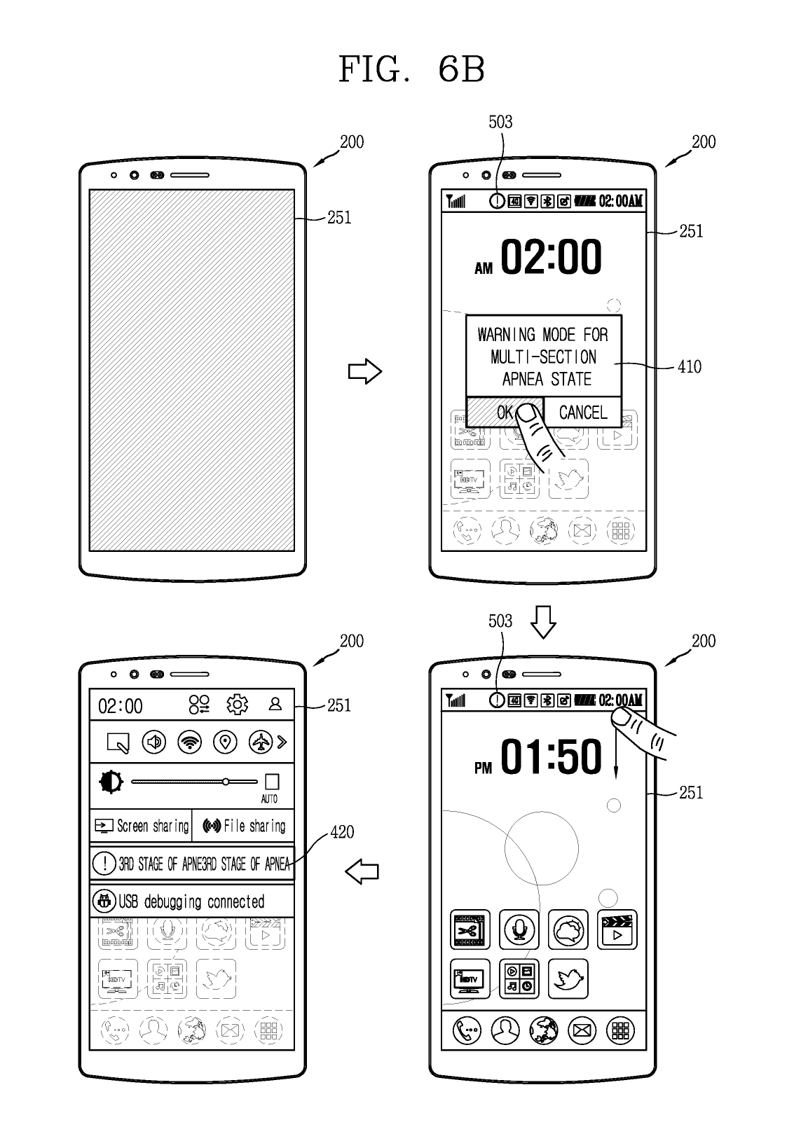

FIG. 6A is a flowchart illustrating a method of controlling a mobile terminal using oxygen saturation detected by a sensing unit of the present invention, and FIG. 6B is a conceptual view illustrating the control method of FIG. 6A. Referring to FIG. 6A, the controller measures oxygen saturation using the sensing unit for a specific time (S11). For example, when the sensing unit detects that the watch-type terminal 100 is worn on the user's wrist, the controller controls the sensing unit to measure the oxygen saturation at preset intervals. Alternatively, the controller may control the sensing unit to measure the oxygen saturation during a specific time of the day, for example, during a sleeping time, while an abnormal state of the body is sensed by another sensor, or while a motion is detected.

The controller analyzes presence or absence of an apnea state using the oxygen saturation (S12). Sleep apnea is a state that breathing is stopped during sleep, which may cause insufficient oxygen to be supplied to the brain, make an autonomic nervous system sensitive, and cause a lack of sleep. Oxygen saturation is reduced due to a lack of oxygen supply in the sleep apnea phase. Therefore, when the calculated oxygen saturation falls below a specific reference value, the controller determines that the user is in the apnea state.

For example, the controller may recognize the apnea state occurred during a sleep time and the number of occurrences of the apnea state, and store information related to the occurrence of the apnea state and the number of occurrences in the memory 170. The controller switches the watch-type terminal 100 to a warning mode and displays a warning mode when the apnea state occurs (or when the apnea state is continued for a predetermined time (or/and has occurred a predetermined number of times) (S13).

Referring to FIG. 6B, the controller switches a mobile terminal to the warning mode when the display unit of the mobile terminal cooperating with the watch-type terminal 100 is activated. In addition, the display unit of the mobile terminal 100 outputs a warning window 410. The warning window 410 may include notification information indicating that the apnea state has occurred in a plurality of sections and the mobile terminal is switched to the warning mode (S13).

When the switching to the warning mode is confirmed, the controller controls the mobile terminal or the watch-type terminal based on the warning mode. However, when the switching to the warning mode is rejected based on a touch input applied on the notification information 410, the controller activates the watch-type terminal or the mobile terminal regardless of the apnea state.

When the user of the watch-type terminal is in the apnea state in multiple sections, a graphic object 503 corresponding to the warning mode may be output on an area (on a status bar) of the display unit of the watch-type terminal or a display unit of an external device cooperating with the watch-type terminal 100. The controller may switch the warning mode to an inactive state based on a touch input applied to the display unit. When the warning mode is switched to the inactive state, the controller may control the mobile terminal and the watch-type terminal regardless of the user's apnea state.

The display unit includes at least one screen information including driving status information based on a drag touch input applied from the status bar, and the screen information includes an image bar 420 corresponding to the warning mode. Although not specifically illustrated, additional information regarding the apnea state may be included on the image bar 420. For example, a time at which the apnea state has occurred, a delay time of the apnea state, pattern information, and the like may be included.

Accordingly, the user can recognize the occurrence of the apnea state during the sleep time (or for a specific time) by the graphic image 503 displayed on the status bar, so as to adjust a physical condition of the user himself/herself. In addition, the user can be provided with detailed information on the apnea state based on an additional touch input applied to the graphic image 503, and recognize the physical condition since the watch-type terminal and the mobile terminal are controlled by the warning mode.

However, the control method according to this embodiment can also be implemented by the watch-type terminal 100. Accordingly, when the apnea state occurs for a predetermined time, the watch-type terminal 100 may not transmit a wireless signal to an external device but be switched to the warning mode.

Hereinafter, a control method of a watch-type terminal 100 or/and a mobile terminal performing wireless communication with the watch-type terminal 100 when the apnea state occurs will be described.

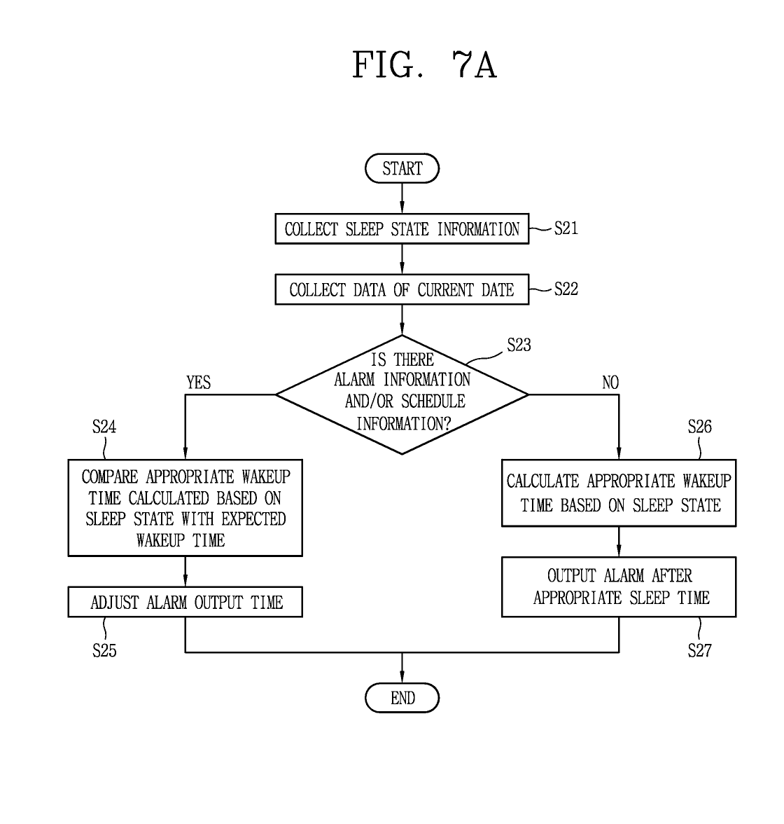

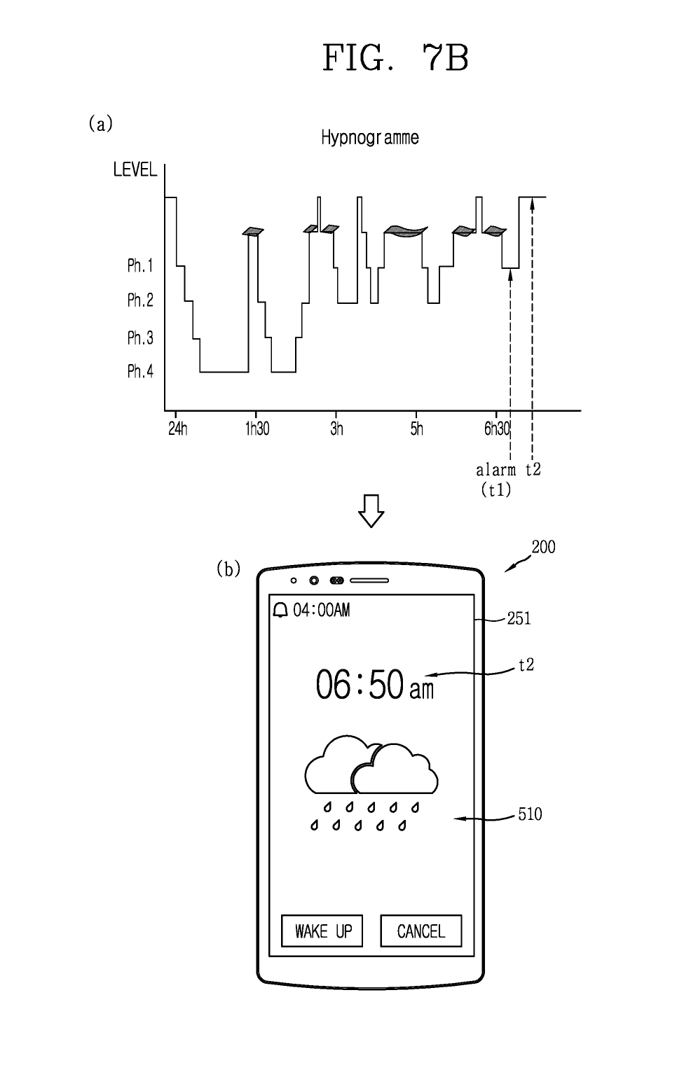

FIGS. 7A and 7B are conceptual views illustrating a method of controlling a watch-type terminal and/or a mobile terminal performing wireless communication with the watch-type terminal, in accordance with one embodiment of the present invention.

Referring to FIG. 7A, the controller 180 collects sleep state information using the sensing unit (S21). Here, the sleep state information may be generated based on occurrence, periodicity, frequency, time, etc. of the sleep apnea state calculated through the oxygen saturation sensed by the sensing unit.

The controller 180 collects data of a current date (S22). For example, data of the current date may include schedule information stored in the current date, weather information related to the current date, information which is related to the current date and received from a server or external device, and the like.

The controller 180 determines whether there is/are alarm information and/or schedule information set by the collected information (S23). When alarm information related to a wakeup time of the current date is collected, the controller 180 compares a calculated proper wakeup time calculated based on the sleep state information with a scheduled wakeup time based on the alarm information (S24), and adjusts an output time of the alarm (S25).

In the adjustment of the output time of the alarm, the controller 180 may analyze and determine history information collected during that time and the user's schedule information. On the other hand, if there is no alarm information or schedule information, the controller 180 calculates an appropriate wakeup time based on the collected sleep state information (S26). The controller 180 outputs the alarm after the appropriate sleep time (S27).

FIG. 7B illustrates a measured sleep level. The sleep level represents a depth of sleep. A lower level corresponds to deeper sleep. When the sleep level is 1 or higher, it corresponds to a REM sleep state in which an activity of the brain is maintained while a muscular activity is stopped. If an alarm output time scheduled by the user's setting is a first time t1, the alarm rings when the user is in a deep sleep state.

In this case, the controller 180 may adjust the alarm time based on a sleep pattern calculated by the oxygen saturation. For example, when the scheduled time for outputting the alarm set by the measured oxygen saturation corresponds to a deep sleep state, the controller 180 may control the alarm to be output at a second time t2 at which the REM sleep state is reached.

When the controller 180 performs wireless communication with an external device, the controller 180 transmits sleep information according to the oxygen saturation to the external device. The external device may adjust the output time by comparing the sleep information with the alarm information. That is, the external device controls an output unit including the display unit to output the alarm information 510 at the second time t2 which is the adjusted output time.

According to this embodiment, the sleep state information can be collected by the oxygen saturation and the output time of the alarm can be changed to a time at which the user is ready to wake up. Thus, it is possible to help the user wake up at an appropriate time based on the user's sleep state.

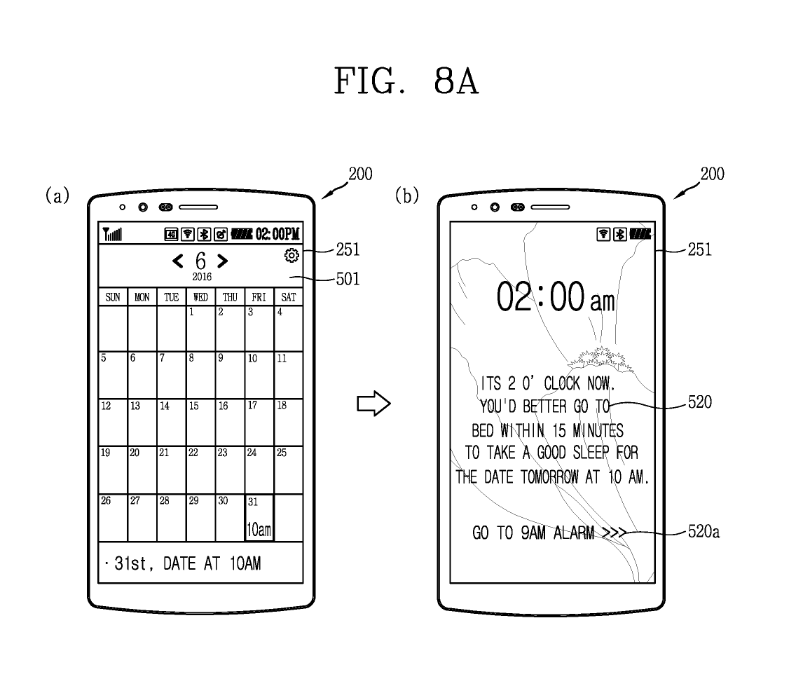

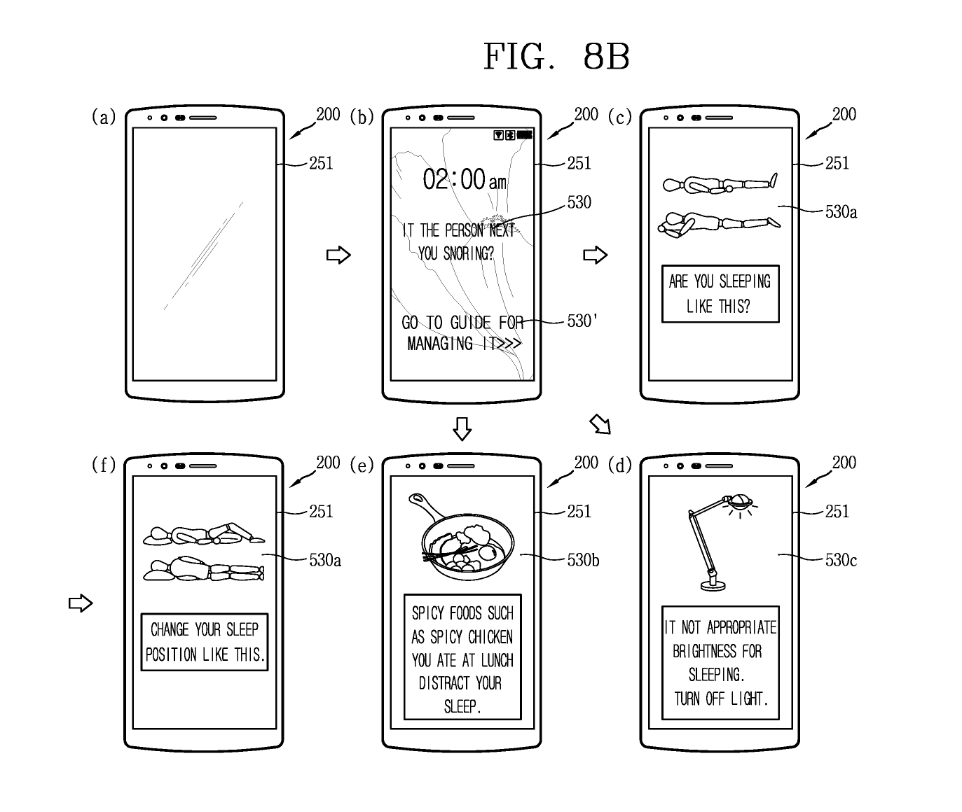

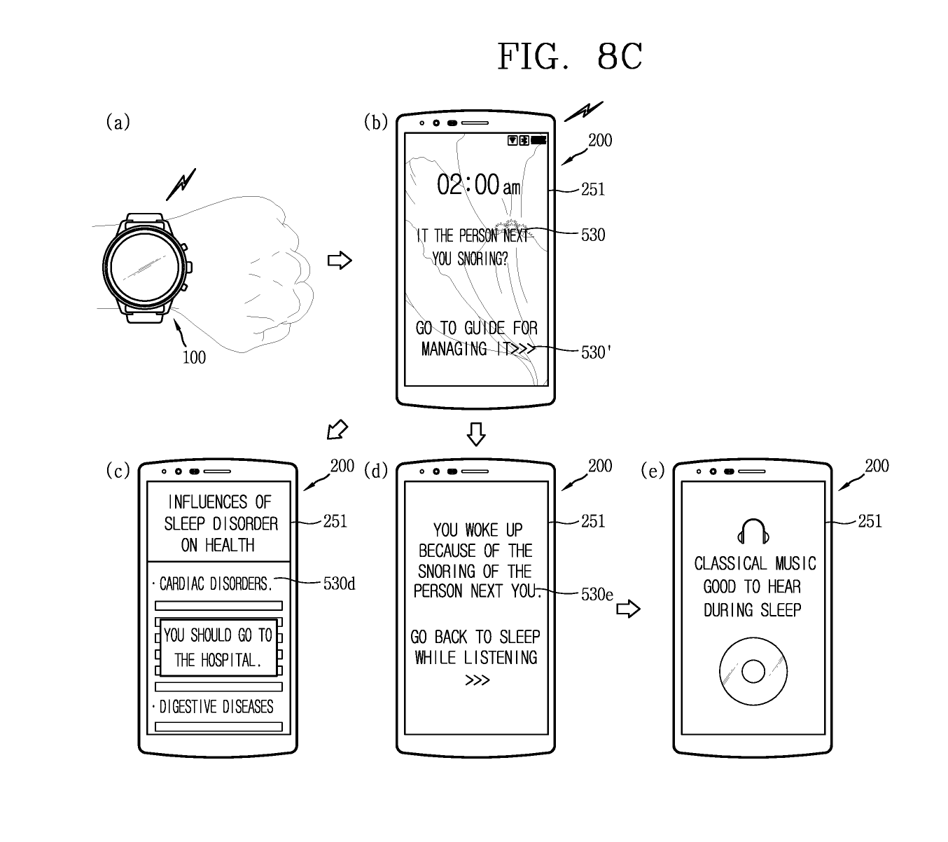

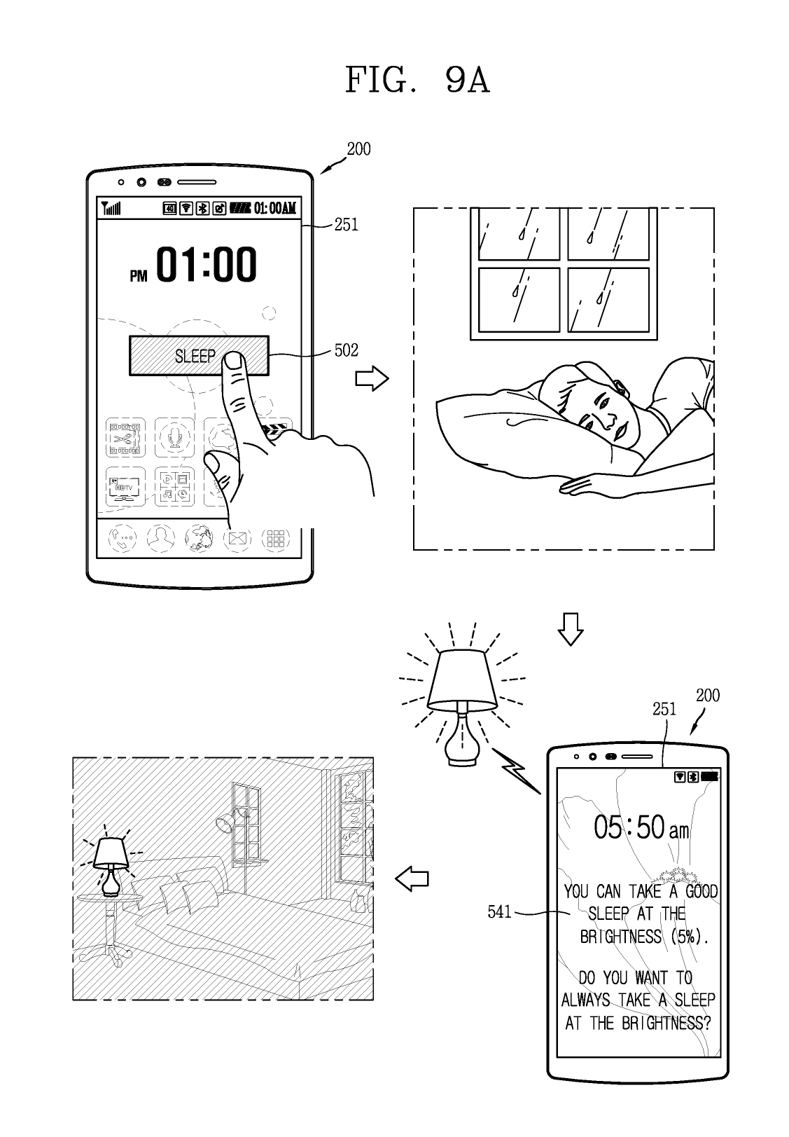

FIGS. 8A to 8C are conceptual views illustrating a control method for providing guide information based on stored information and sleep state information. FIGS. 8A to 8C illustrate one example of an external device that performs wireless communication with the watch-type terminal 100 of the present invention. However, such a control method may be equally applied to the watch-type terminal 100 of the present invention.

Referring to FIG. 8A, schedule information may be stored in the memory of the external device or the memory 170 of the watch-type terminal 100 based on the user's control command. (a) of FIG. 8A shows first screen information 501 including the schedule information. The controller 180 may calculate an appropriate sleep time of the user based on sleep state information stored in the memory 170. The controller 180 may output first guide information 520 guiding the user's sleep based on a current time, and the sleep state information and the schedule information stored in the memory 170.

The guide information 520 may be displayed on the display unit 151 of the watch-type terminal 100 or may be displayed on a display unit of the external device performing wireless communication with the watch-type terminal 100. In this case, the guide information 520 may include a control image 520a that receives a touch input for setting a new alarm. Although not specifically illustrated, when a touch input is applied to the control image 520a, an application for setting an alarm may be executed.

The guide information 520 may be implemented by auditory data or vibration as well as visual data. For example, the guide information 520 may include information related to a time to start sleeping by comparing an appropriate sleep time with a current time, or may be implemented as text and/or image indicating information related to a time at which the user can sleep and information related to a prestored schedule.

Referring to FIG. 8B, the external device performs wireless communication with the watch-type terminal 100 having the sensing unit, and transmits the selected sleep state information to a preset specific external device or an external device located within a specific range. The external device which has received the sleep state information may correspond to an external device of another user who is different from the user of the watch-type terminal 100.

If the sleep state information does not correspond to a normal sleep state range, the controller 180 of the watch-type terminal 100 transmits the sleep state information to an adjacent external device. For example, when the apnea state occurs due to snoring or the apnea state is frequently detected, specific information is transmitted to the user' mobile terminal adjacent to the watch-type terminal 100.

The external device which has received the sleep state information through the wireless communication with the watch-type terminal 100 outputs second guide information 530 based on the sleep state information. The second guide information 530 may be visual data displayed on the display unit of the mobile terminal, or may be realized as auditory data or vibration. A control image 530′ for providing additional information may be included when the second guide information 530 corresponds to the visual data.

Additional guide information may be output based on a touch input applied to the control image 530′. First additional guide information 530a includes information related to a sleep position of the user of the watch-type terminal 100, and second additional guide information 530b provides an analysis result by extracting information stored in the watch-type terminal 100. For example, the second additional guide information 530b may include guide information for restraining an intake of food while providing food intake information stored in the watch-type terminal 100. Meanwhile, third additional guide information 530c provides an analysis result using sensing information sensed by a sensor unit mounted on the external device that outputs the guide information. For example, the third additional guide information 530c may include guide information for adjusting lighting through illuminance sensed through an illuminance sensor of the external device.

Referring to FIG. 8C, additional guide information according to another embodiment will be described. The external device is set to perform wireless communication with the watch-type terminal 100. The external device may output guide information 530 including the control image 530′ to the display unit when the sleep state information is received from the watch-type terminal 100.

The watch-type terminal 100 may transmit the guide information together with the sleep state information to the external device. For example, the watch-type terminal 100 may transmit the sleep state information together with health state information of the user to the external device. Accordingly, the external device outputs fourth additional guide information 530d including the sleep state information and the health state information. Accordingly, the user of the external device can take an appropriate action to the user through the fourth additional guide information 530d.

On the other hand, the external device displays fifth additional guide information 530e recommending a preset function based on the sleep state information. For example, the set function may correspond to a music playback function which is helpful for sleeping. The controller 180 of the watch-type terminal 100 may simultaneously transmit a control command for causing the specific function to be executed, when transmitting the sleep state information. Accordingly, a specific function that helps the sleeping state can be executed based on the control command before the user of the external device wakes up.

According to the embodiments of the present invention, guide information can be directly provided to the user wearing the watch-type terminal 100 and also provided through an adjacent external device which performs wireless communication with the watch-type terminal. This may help the user to sleep by providing information to another person without waking up the user of the watch-type terminal 100 who is sleeping.

Accordingly, the present invention provides a function which is helpful for the sleep state of another person as well as the user. However, the guide information and the additional guide information may be directly output by the watch-type terminal 100.

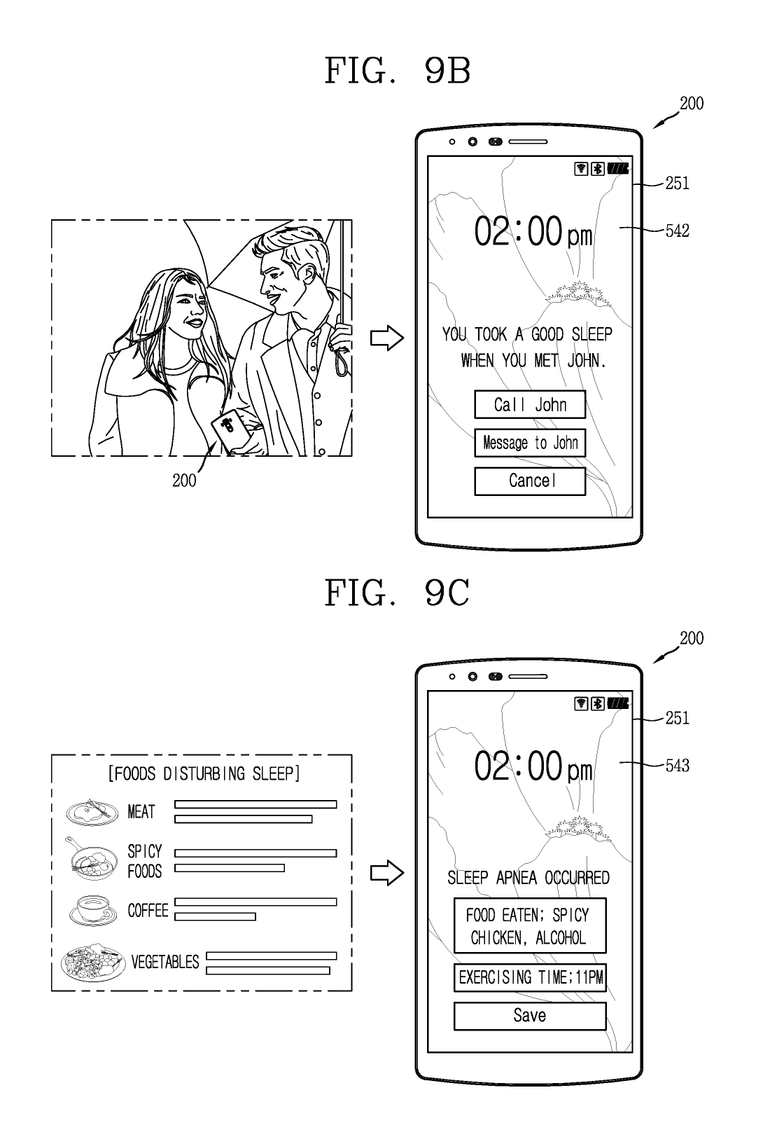

FIGS. 9A to 9C are conceptual views illustrating a control method for providing guide information analyzed through collected sleep state information and additional information. The guide information according to this embodiment may be output directly by the watch-type terminal 100 or may be output by an external device which receives the sleep state information from the watch-type terminal 100.

Referring to FIG. 9A, a sleep mode may be activated based on a control command of the user. For example, the control command of the user may be generated based on a touch input applied to a control image 502 displayed by the external device, or may be transmitted by the watch-type terminal 100.

The external device may detect external brightness when the sleep mode is activated. Alternatively, the watch-type terminal 100 may control the sensor unit to detect the external brightness in the sleep mode, and transmit the result to the external device.

If it is detected that the user is not in a good sleep state based on the sleep state information, first control guide information 541 is output. The first control guide information 541 includes guide information for adjusting the external brightness. When there is a lighting which cooperates with the watch-type terminal 100, the watch-type terminal may transmit a wireless signal to the lighting to lower brightness.

Based on the sleep state information, the external device and the watch-type terminal 100 may detect the external brightness and transmit a wireless signal to adjust brightness of the lighting such that the brightness is similar to the external brightness.

Referring to FIG. 9B, the watch-type terminal forms second control guide information 542 based on analysis results of the sleep state information and storage information related to a date on which the sleep state information was collected. The second control guide information 542 may include a graphic image for causing a specific function to be executed based on the storage information. For example, positive data is collected through recorded log information of the day when the user took a good sleep based on the sleep state information, and negative data is collected through recorded log information of the day when such a good sleep was not taken.

For example, when a schedule that the user met a specific person on the day when the user took a good sleep has been stored or when there is log information related to data transmission and reception with a specific external device, the second control guide information 542 may include a graphic image for performing a wireless communication function with the specific person or the external device.

Referring to FIG. 9C, the watch-type terminal 100 may store intake information related foods that the user ate together with the sleep state information. The controller 180 analyzes an association result based on the sleep state information and the food intake information. For example, if the good sleep was not taken based on the sleep state information, the foods included in the intake information of the day are collected as negative data.

Accordingly, third control guide information 543 may include visual data indicating the intake of the food should be avoided, while providing food information that the user ate on the day when the sleep apnea occurred.

The first to third control guide information 541, 542 and 543 may be displayed on the display unit 151 of the watch-type terminal 100 or may be displayed on the external device performing the wireless communication with the watch-type terminal 100.

That is, the user may analyze sleep state information, which includes information on whether or not the apnea has occurred during sleep, frequency of occurrence, an occurrence duration, an occurrence time of the sleep apnea, etc., together with log information of another user, thereby obtaining guide information for a better sleep state. Therefore, the user does not have to consciously analyze the sleep state and his/her behavior.

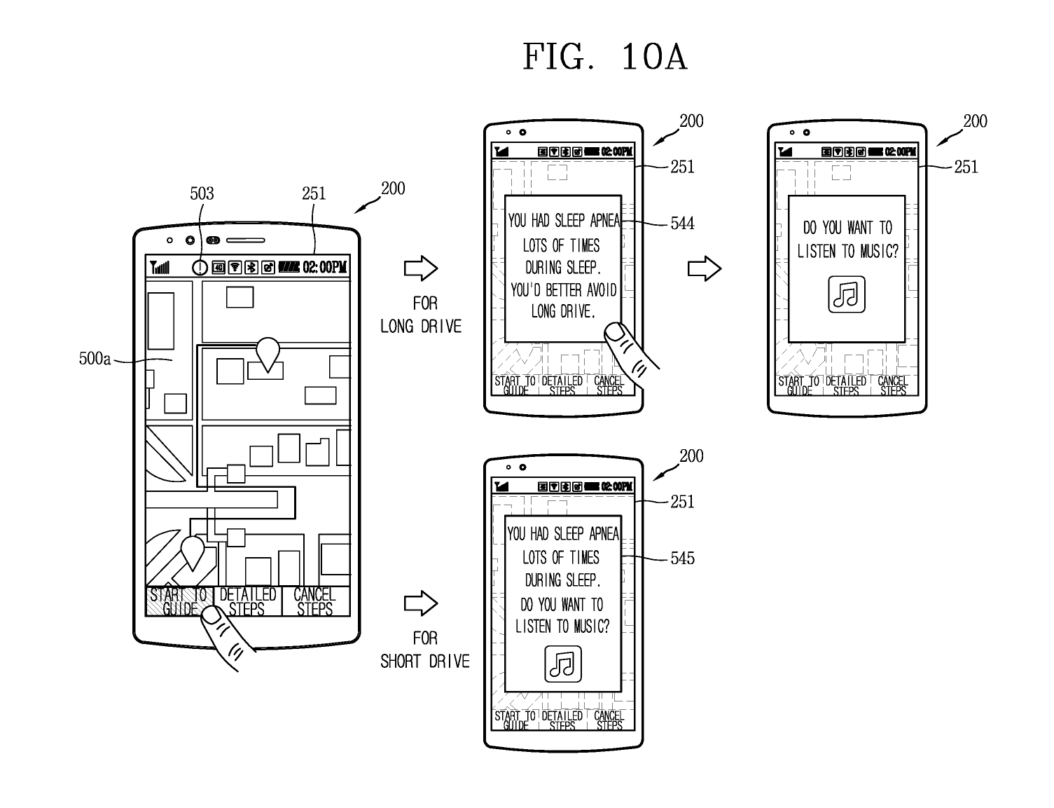

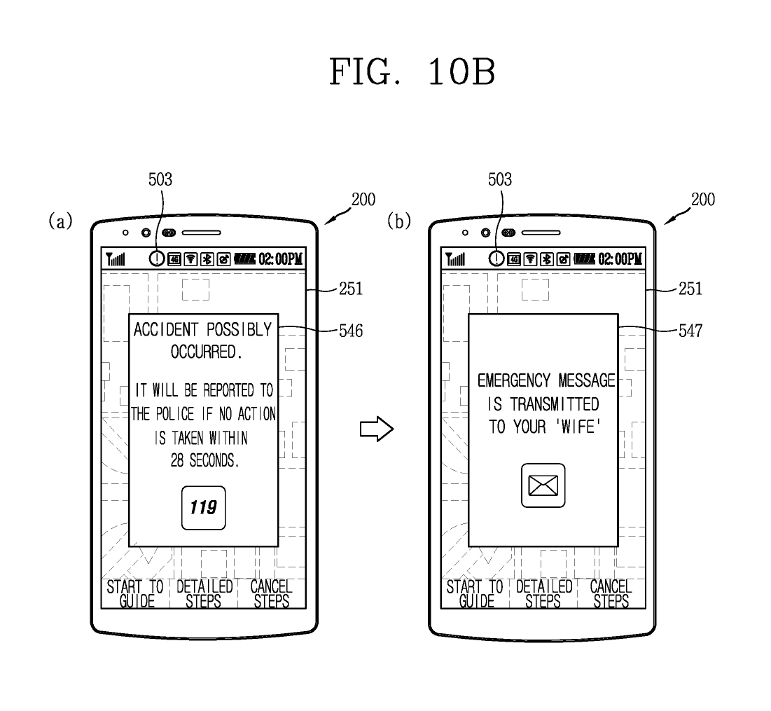

FIGS. 10A to 10C are conceptual views illustrating a control method in a state where a warning mode is activated. Referring to FIGS. 10A and 10B, when it is determined that a sleep state is not good by a preset reference based on the sleep state information, the watch-type terminal 100 activates a warning mode and/or transmits a wireless signal to an external device cooperating with the watch-type terminal 100 such that the warning mode is also activated in the external device. That is, although the drawings are given to explain a control method of a mobile terminal as an external device, but the present invention is not limited thereto, and the watch-type terminal 100 may be driven or controlled in substantially the same manner.

Referring to FIG. 10A, when the warning mode is activated, the display unit of the external device outputs an icon 503 informing it. The sleep state information may be displayed in detail or the warning mode may be released based on a touch input applied to the icon 503.

The display unit displays an execution screen 500a corresponding to an executed specific function. When the specific function is executed by the execution screen 500a, a first warning window 544 corresponding to the specific function is displayed. The first warning window 544 may include a message for confirming whether the specific function is executed, but the present invention is not limited thereto. The first warning window 544 includes text for explaining why the execution is restricted, while restricting the execution of the specific function. Or, the first warning window 544 may include only warning text to stop the execution of the specific function while maintaining the execution of the specific function. After the output of the first warning window 544, a control window of another function that can be executed together with the specific function may be displayed.

On the other hand, the watch-type terminal 100 and the external device may output a second warning window 545 based on the execution of the specific function in the warning mode. The second warning window 545 may include a guide message for executing a function that can be executed together with the specific function. For example, the second warning window 545 may include text to guide an execution of a music playback application.

When the same application is activated, the external device or the watch-type terminal 100 may selectively output the first warning window 545 or the second warning window 545 based on an executed function and a condition of the executed function.

Referring to FIG. 10B, when a specific change is detected by the external device or the watch-type terminal while the warning mode is being executed, a third warning window 546 is output. The third warning window 546 may be displayed on the watch-type terminal 100 or on the external device. The specific change may correspond to a sudden change in an acceleration state. The third warning window 546 may include a message indicating that it will be determined as an occurrence of a failure or accident when a signal is not applied based on the sudden change.

If a requested signal input is not applied after the third warning window 546 is output, a fourth warning window 547 may be displayed. The fourth warning window 547 includes a guide message indicating that information related to the specific change is transmitted to another external device. In this case, the information on the specific change may be transmitted to an external device which frequently performs wireless communication with the external terminal or the watch-type terminal or may be transmitted to a preset external device.

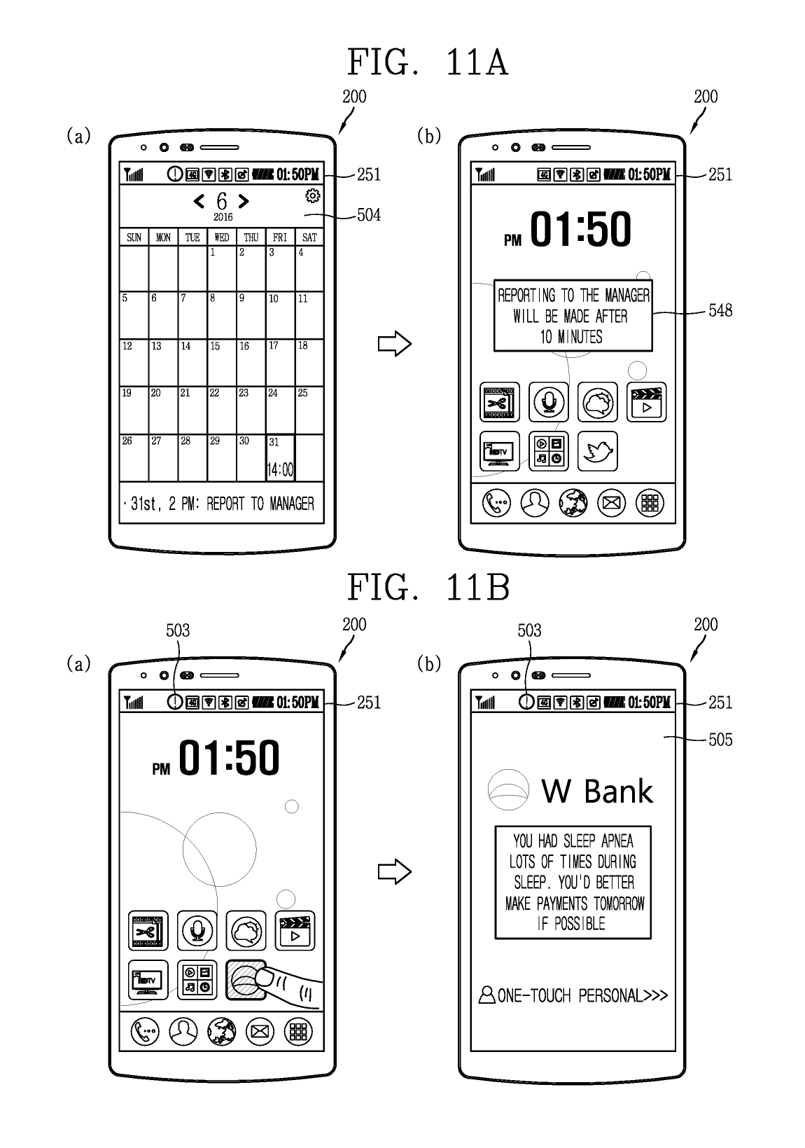

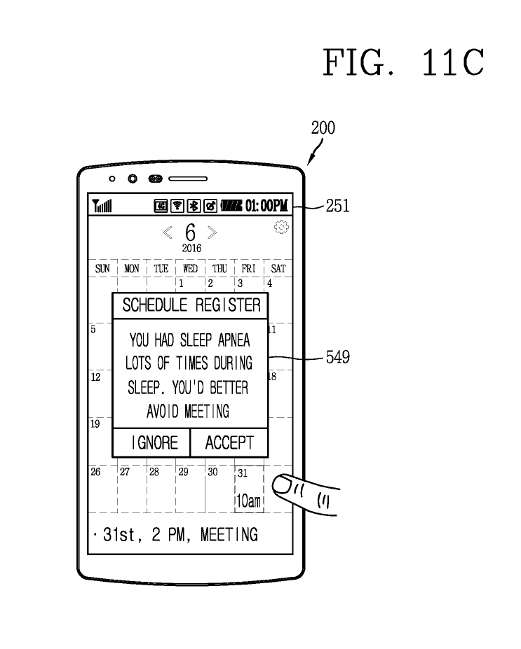

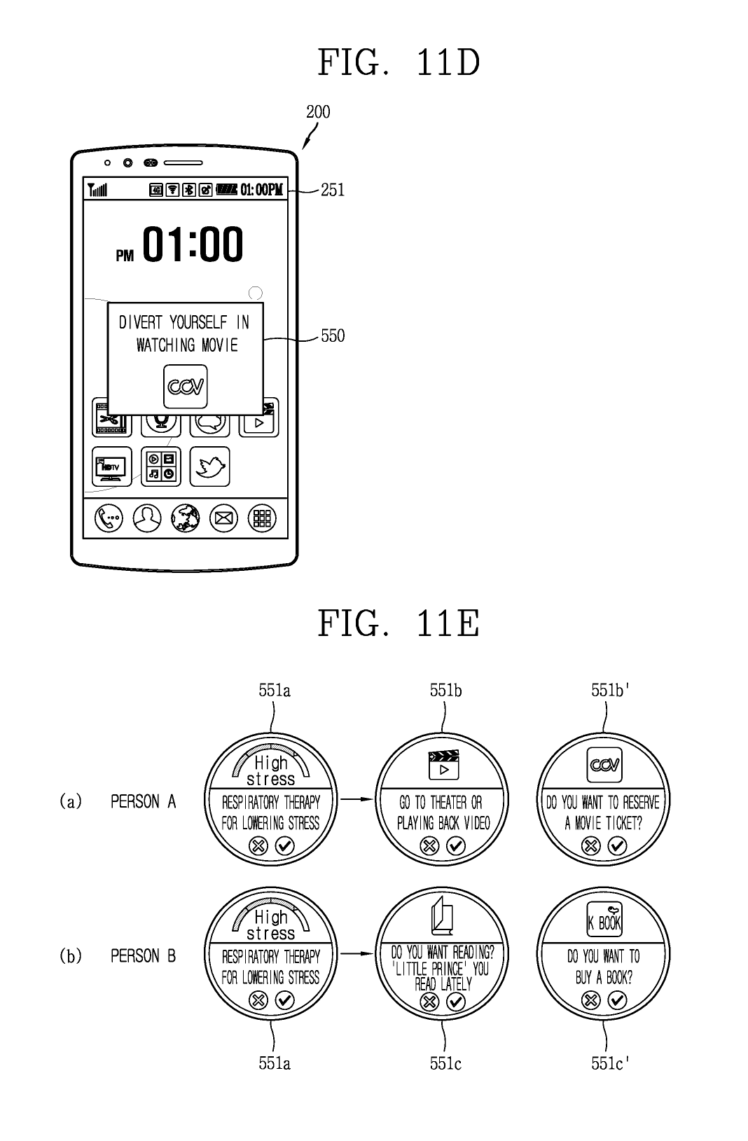

FIGS. 11A to 11E are conceptual views illustrating a control method of a watch-type terminal and an external device cooperating with the watch-type terminal according to another embodiment. Although the mobile terminal is shown in the drawing, the control method may be applied to the watch-type terminal in the same manner, and thus a duplicate explanation will be omitted.

Referring to FIG. 11A, the watch-type terminal 100 recognizes oxygen saturation and a sleep apnea state through the sensor unit. When the sleep state information is generated, the watch-type terminal 100 may transmit the sleep state information to an external device. Alternatively, the watch-type terminal 100 may transmit the sleep state information to the external device when the sleep state of the user is unstable.

If schedule information is stored in the external device through a specific application 504, an alarm 548 for the schedule information is output based on the sleep state information. The external device may output the alarm 548 at more frequent intervals when the sleep state information is received.

On the other hand, when sleep state information indicating an unstable sleep state is generated, the watch-type terminal 100 may output a notification informing the stored schedule information, or may output an alarm about the schedule information more frequently. According to this embodiment, the user who has a chance of a failure of memory due to an unstable sleep may be notified not to miss a prestored schedule.

Referring to FIG. 11B, if the sleep state information indicating the unstable sleep state is received by the external device, the external device may output a warning screen 505 when an application related to security is executed. For example, the security-related application may correspond to an application associated with financial operations, or the like. If the sleep state information indicating the unstable sleep state is generated, the watch-type terminal 100 may output a warning screen on the display unit 151 when the security-related application is executed in the watch-type mobile terminal 100.

Referring to FIG. 11C, when the sleep state information indicating the unstable sleep state is received by the external device, the external device may output a warning message 549 based on recorded schedule information. The warning message 549 may include the user's sleep state. On the other hand, when the sleep state information indicating the unstable sleep state is generated, the watch-type terminal 100 may display a warning message corresponding to the schedule information stored in the memory 170 on the display unit 151.

Referring to FIG. 11D, when the sleep state information indicating the unstable sleep state is received by the external device, the external device may output behavior guide information 550 of the user. For example, watching movies, reading, and exercising may be recommended for the user's diversion.

Meanwhile, when the sleep state information indicating the unstable sleep state is generated, the watch-type terminal 100 may output behavior guide information. As a result, it is possible to improve the mood of the user who feels uneasy and depressed due to an insufficient sleep.

Referring to FIG. 11E, the display unit 151 of the watch-type terminal 100 may display an image 551a representing a stress index based on the sleep state information, and output first screen information 551b for recommending watching movies or second screen information 551c for recommending reading. Also, the display unit 151 of the watch-type terminal 100 may output first and second execution guide screens 551b′ and 551c′ for taking an action based on the first and second screen information 551b and 551c. The controller 180 may guide a behavior or action required for the user to take by analyzing behavior log and approval information of the user stored in the memory 170, and the sleep state information.

The present invention can be implemented as computer-readable codes in a program-recorded medium. The computer-readable medium may include all types of recording devices each storing data readable by a computer system. Examples of such computer-readable media may include hard disk drive (HDD), solid state disk (SSD), silicon disk drive (SDD), ROM, RAM, CD-ROM, magnetic tape, floppy disk, optical data storage element and the like. Also, the computer-readable medium may also be implemented as a format of carrier wave (e.g., transmission via an Internet). The computer may include the controller 180 of the terminal. Therefore, it should also be understood that the above-described embodiments are not limited by any of the details of the foregoing description, unless otherwise specified, but rather should be construed broadly within its scope as defined in the appended claims, Therefore, all changes and modifications that fall within the metes and bounds of the claims, or equivalents of such metes and bounds are therefore intended to be embraced by the appended claims.

The present invention provides a watch-type terminal for sensing a breathing state by disposing a red light-emitting element for outputting red light and an IR sensor to be spaced apart from a light-receiving sensor by a specific distance or more. Therefore, the present invention can be utilized in various related industrial fields.

1-20. (canceled)

21. A watch-type terminal, comprising:
a main body;
a sensing unit disposed on the main body;
a display unit; and
a controller,
wherein the sensing unit comprises:
at least one green light-emitting element to output green light;
a light-receiving sensor spaced apart from the at least one green light-emitting element to receive green light reflected from one part of the human body;
a red light-emitting element spaced apart from the light-receiving sensor to output red light; and
an infrared (IR) sensor spaced apart from the light-receiving sensor to output IR light, and
wherein the controller calculates an oxygen saturation of blood in a human body wearing the watch-type terminal based on an oxygen absorbance of hemoglobin in the human body through reflectance of the red light and the IR light.

22. The terminal of claim 21, wherein a spaced distance between the light-receiving sensor and the at least one green light-emitting element is a first length, and a spaced distance between the light-receiving sensor and the red light-emitting element and the IR sensor is a second length longer than the first length.

23. The terminal of claim 21, wherein the controller calculates breathing state information related to an apnea state of the human body based on the calculated oxygen saturation.

24. The terminal of claim 23, wherein the controller activates a warning mode related to an execution of a specific function based on the calculated breathing state information.

25. The terminal of claim 24, wherein the controller restricts the execution of the specific function or displays guide information recommending a function executable together with the specific function on the display unit, in the warning mode.

26. The terminal of claim 25, further comprising:
a memory to store schedule information,
wherein the controller calculates an appropriate sleep time based on the calculated breathing state information, and changes an output time of notification information notifying the schedule information based on the calculated appropriate sleep time.

27. The terminal of claim 26, wherein the controller executes a preset application or displays guide information for guiding the execution of the preset application on the display unit, based on the calculated breathing state information.

28. The terminal of claim 25, wherein the controller changes the output time when a sleep state of the output time of the notification information corresponds to a deep sleep state based on the calculated breathing state information.

29. The terminal of claim 25, wherein when the specific function is executed in the warning mode, the controller displays guide information on the display unit including a message restricting the execution of the function or a message recommending an execution of another function.

30. The terminal of claim 25, further comprising:
a sensor,
wherein the controller displays a warning window associated with an occurrence of a failure or executes a function of transmitting information related to the occurrence of the failure to an external device when a specific change is detected by the sensor.

31. The terminal of claim 24, further comprising:
a wireless communication unit to perform wireless communication with a preset external device,
wherein the controller controls the wireless communication unit to transmit the calculated breathing state information to the preset external device.

32. The terminal of claim 31, further comprising:
a memory to store log information,
wherein the controller transmits log information stored in the memory together with the calculated breathing state information.

33. The terminal of claim 32, wherein the controller generates guide information for guiding a user's behavior based on the log information and the breathing state information, and
wherein the log information includes at least one of schedule information, food intake information, and log information of the wireless communication.

34. The terminal of claim 25, wherein the controller displays a graphic image corresponding to the warning mode on the display unit when switched to the warning mode.

35. The terminal of claim 34, wherein the controller releases the warning mode or displays the calculated breathing state information when a touch input is applied to the graphic image.

36. The terminal of claim 21, wherein the at least one green light-emitting elements includes two green light-emitting elements, and
wherein the two green light-emitting elements, the red light-emitting element, and the IR sensor surround the light-receiving sensor or are arranged along one direction.

37. The terminal of claim 21, wherein the light-receiving sensor includes two light-receiving sensors.

38. A method for controlling a watch-type terminal, the method comprising:
measuring, via a sensing unit of the watch-type terminal, oxygen saturation of a human body wearing the watch-type terminal;
analyzing, via a controller of the watch-type terminal, whether an apnea state occurs based on the oxygen saturation;
calculating, via the controller, breathing state information based on the oxygen saturation;
activating, via the controller, a warning mode based on the breathing state information; and
executing, via the controller, a function executed by a control command in the warning mode,
wherein the sensing unit comprises:
at least one green light-emitting element to output green light;
a light-receiving sensor spaced apart from the at least one green light-emitting element to receive green light reflected from one part of the human body;
a red light-emitting element spaced apart from the light-receiving sensor to output red light; and
an infrared (IR) sensor spaced apart from the light-receiving sensor to output IR light, and
wherein the measuring measures the oxygen saturation of blood in the human body based on an oxygen absorbance of hemoglobin in the human body through reflectance of the red light and the IR light.

39. The method of claim 38, further comprising transmitting the calculated breathing state information to a preset external device.

40. The method of claim 38, further comprising:
displaying guide information on a display unit of the watch-type terminal based on prestored information and the calculated breathing state information,
wherein the prestored information includes at least one of schedule information, alarm information, and wireless communication log information.
